(19) United States
(12) Patent Application Publication
(10) Pub. No.: US 2026/0224707 A1
(43) Pub. Date: Aug. 6, 2026

(54) Compositions and Methods to Achieve Long-Acting Sustained Release of Hydrophobic Small Molecule Drugs and Methods of Making and Using

(71) Applicant: Brown University, Providence, RI (US)

(72) Inventors: Edith Mathiowitz, Brookline, MA (US); Cameron Baptista, Phillipsburg, NJ (US); El Hadji Arona Mbaye, Providence, RI (US); Anyaa Shah, Providence, RI (US); Apoorva Nagaraj Rao, Providence, RI (US); Zihao Chen, Providence, RI (US); Rudra Trivedi, Providence, RI (US); Raj Sharan, Providence, RI (US)

(21) Appl. No.: 19/465,926

(22) Filed: Jan. 30, 2026

Related U.S. Application Data
(60) Provisional application No. 63/752,237, filed on Jan. 31, 2025.

(51) Int. Cl. A61K 47/34; A61K 9/70; A61K 31/12; A61K 31/5365; A61K 31/5415; A61K 31/573; A61K 47/32

(52) U.S. Cl. CPC A61K 47/34; A61K 9/7007; A61K 31/12; A61K 31/5365; A61K 31/5415; A61K 31/573; A61K 47/32

(57) ABSTRACT

Described are compositions for controlled long-term release of hydrophobic drugs in subjects. The compositions contain a polymer matrix containing a hydrophobic polymer and a hydrophobic drug dispersed within the polymer matrix, with a mesophase content between about 5% and about 40%. The compositions disclosed herein allow for the loading of a wide variety of hydrophobic drugs within the polymer matrix. Also described, are methods for making these compositions, by inducing mesophase formation in a polymer matrix containing a hydrophobic polymer and a hydrophobic drug. The methods include: (i) heating a combination of the hydrophobic polymer and the hydrophobic drug to a first temperature that is less than the melting temperature of the hydrophobic polymer, (ii) applying to the combination an elevated pressure greater than atmospheric pressure and at the first temperature, and (iii) cooling the combination to a desired temperature, while maintaining the elevated pressure in step (ii).

### CROSS-REFERENCE TO RELATED APPLICATIONS

This application claims the benefit of and priority to U.S. Provisional Application No. 63/752,237 filed on Jan. 31, 2025, the contents of which is incorporated herein in its entirety.

### FIELD OF THE INVENTION

This invention is generally in the area of compositions for delivery of hydrophobic small molecule drugs.

### BACKGROUND OF THE INVENTION

Long-term drug delivery administration techniques have been dominated by injection as the primary tool for administering drugs in clinical settings. This can be attributed to the usually higher bioavailability of injectable drugs compared to other administration methods and technologies. However, when a patient is injected, the drug follows a pattern of release in the body characterized by peaks and valleys of drug in the blood plasma of the patient. These peaks and valleys of drug concentration in the blood may fall above and below the desired concentration range of drug in the patient's blood, often causing the patient to receive multiple doses of the drug and creating an unstable effect of the drug uptake over time.

An unstable uptake of drug over time and increasing number of doses for a patient can make patients less adherent to drug dosages and schedules, especially for patients that must take multiple drugs daily because of their chronic conditions. Furthermore, drugs administered through injection can cause toxicity in a patient, due to the high concentration of drug being released into the patient's system. Patients that are not able to have their drugs routinely injected are often limited to the oral administration of drugs. Because the oral administration of drugs generally has a lower bioavailability than that of injections, patients are forced to take multiple doses daily or weekly to continue the desired effect of the drug on their symptoms. An unstable uptake of drug over time and increasing number of doses for a patient can again reduce patient drug adherence. Additionally, patients may be prone to drug abuse when administered orally, further reducing the desired therapeutic effect of the drug and in some cases raising the morbidity rate of the patient population. Accordingly, there remains a need for improved compositions for long-term delivery of drugs.

Therefore, it is an object of the invention to provide improved compositions for drug delivery.

It is a further objection of the invention to provide compositions for delivery of hydrophobic drugs.

It is a further objection of the invention to provide compositions for long-term delivery of hydrophobic drugs.

It is another object of the invention to provide methods for making compositions for improved delivery of hydrophobic drugs.

### SUMMARY OF THE INVENTION

Described are compositions for controlled long-term release of hydrophobic drugs in subjects in need thereof. The compositions contain a polymer matrix containing a hydrophobic polymer and/or an amphiphilic polymer, and a hydrophobic drug dispersed within the polymer matrix. The polymer matrix has a mesophase content between about 5% and about 40% as measured using X-ray diffraction techniques at atmospheric pressure and room temperature (between 20° C. and 30° C.). The hydrophobic polymer is a biodegradable polymer, preferably a biodegradable thermoplastic polymer. A preferred class of hydrophobic polymers includes a polyester, preferably a linear aliphatic polyester. Specific examples of these polymers include a poly(lactic acid), poly(caprolactone), poly(lactic-co-glycolic acid). In some forms, the mesophase is formed from a blend of two or more hydrophobic polymers, such as two or more types of polyesters. Examples of blends include poly(lactic acid) and poly(caprolactone), poly(lactic acid) and poly(lactic-co-glycolic acid), or poly(caprolactone) and poly(lactic-co-glycolic acid).

In some forms, the polymer matrix further contains a biocompatible polymer having a non-biodegradable backbone, or preferably a biocompatible, non-biodegradable polymer. Examples of these polymers include polyvinyl alcohol; polyalkylene oxides (e.g., poly(ethylene glycol), poly(ethylene oxide), poly(propylene oxide), and copolymers thereof, such as poloxamers); polymethacrylates; and copolymers of these polymers.

The compositions disclosed herein allow for the loading of a variety of one or more hydrophobic drugs within the polymer matrix. Preferably, the one or more hydrophobic drugs are homogeneously distributed within the polymer matrix, and not covalently bound to a polymer (e.g., a hydrophobic polymer) that forms the polymeric matrix.

In some forms, the one or more hydrophobic drugs have a loading between about 1% w/w and about 60% w/w, about 2.5% w/w and about 60% w/w, about 5% w/w and about 60% w/w, about 1% w/w and about 50% w/w, about 2.5% w/w and about 50% w/w, about 5% w/w and about 50% w/w, 1% w/w and about 25% w/w, of the polymeric matrix. In some forms, the one or more hydrophobic drugs have a loading between about 1% w/w and about 60% w/w, about 1% w/w and about 50% w/w, or about 1% w/w and about 25% w/w, of the composition. Without wishing to be bound by theory, it is believed that inducing a mesophase alters the diffusion properties of drugs within the polymer matrix, and those alter release kinetics, where diffusion is the rate-limiting factor. In some forms, the hydrophobic drug is released from the composition between one and 100 days, inclusive, between one and 180 days, or up to one year.

Also described, are methods for making these compositions. The methods involve inducing mesophase formation in compositions containing a polymer matrix containing a hydrophobic polymer and/or amphiphilic polymer, and a hydrophobic drug. The methods include the steps of step (i) heating a combination of the hydrophobic polymer and/or amphiphilic polymer, and the hydrophobic drug to a first temperature that is less than the melting temperature of the hydrophobic polymer and/or amphiphilic polymer, step (ii) applying to the combination an elevated pressure greater than atmospheric pressure and at the first temperature between about 5 mins and 15 mins, and step (iii) cooling the combination to a desired temperature, while maintaining the elevated pressure in step (ii). In some forms, the methods further involve applying to the combination a first pressure prior to step (i). Preferably, the first pressure is between 50 MPa and 275 MPa, inclusive, or 50 MPa and 140 MPa, such as about 65 MPa, about 66 MPa, about 67 MPa, about 68 MPa, about 69 MPa, or about 70 MPa. Preferably, the elevated pressure is between about 14 MPa and about 275 MPa, about 14 MPa and about 140 MPa, such as about 125 MPa, about 130 MPa, about 135 MPa, about 138 MPa, or about 140 MPa. In some specific forms, the first pressure is about 69 MPa, and the elevated pressure is about 138 MPa. In some forms, the heating in step (i) is performed in a mold at a suitable temperature to enhance fusion of the composition, i.e., melt casting the combination.

Alternatively, the methods for inducing the mesophase involve first performing a film casting a composition containing the hydrophobic polymer and/or amphiphilic polymer, and the hydrophobic drug, and then applying stress to the film, such as stretching the film.

Before applying pressure or stress in each case, preferably, there is an initial preparation step that involves mixing the polymer and the drug together. Mixing the polymer and the drug can depend on their physical properties. If both the polymer and the drug are solid particles, simple physical mixing can be done. Alternatively, both the drug and polymer can be mixed and dissolved in a solvent that is suitable for both, followed by film casting and solvent evaporation. Another method involves dissolving the polymer in a suitable solvent and then dispersing the drug. Lastly, the polymer and drug can be mixed, melted, and then extruded together. In this final method, care is taken not to damage the drug. In sum, one can form any delivery systems and then apply the pressure. In the case where the delivery systems are microparticles, for example, pressure can be applied after formation of the microparticles in a pressure vessel. In such an instance, appropriate measures are taken to prevent compromising the shape and/or size of the microparticles. The drug and polymer must be mixed before applying the steps described to induce the mesophase.

The disclosed compositions can be used as a versatile platform for delivery of one or more hydrophobic drugs, preferably long-term delivery. Given their demonstrated long-term release kinetics, the disclosed compositions can find application in chronic diseases or disorders that require patients to receive multiple doses of one or more drugs. The compositions can be administered tablets or implants (such as films, membranes, fibers, or cylinders). The films, membranes, fibers, or cylinders can be single-layered, multi-layered, unidirectional (i.e., release drug through only side), multi-directional (i.e., release drug through at least two sides), or a combination thereof. Preferably, the compositions are administered in a solid form.

### BRIEF DESCRIPTION OF THE DRAWINGS

FIG. 1 is a schematic showing different phases of polymers, including the semi-crystalline and amorphous states. Mesophases are found between these states in the forms of plastic crystals, condis crystals, and liquid crystals.

FIG. 2 is a schematic showing polymer sample processing methods, including a second press processing method. In the second press processing method, using a temperature-controlled hydraulic press, both pressure and temperatures of 25° C. and 250° C. can be applied to samples. This temperature range will change according to the type of polymer used. In general, the mesophase can be induced below the glass transition and up to below the melting point of the specific polymers. For PCL the melting point is 60° C. and the glass transition −60° C. for PLA the meting point is about 174° C. and the glass transition is 64° C. Each polymer that has different melting and glass transition points will require different processing conditions.

FIGS. 3A-3D are line graphs comparing PCL X-ray powder diffraction (XRD) data amongst day 0 samples. XRD analysis and comparison of each processing method used on (FIG. 3A) blank PCL, (FIG. 3B) PCL with 5% w/w meloxicam (MLX) loading, (FIG. 3C) PCL with 10% w/w MLX loading, and (FIG. 3D) PCL with 20% w/w MLX loading. All data was analyzed for day 0 samples. Curves were normalized for area to compare peak shape more accurately across different processing methods.

FIG. 4. is a bar graph showing mesophase content of PCL, MLX loaded samples at day 0. Mesophase content was taken for each MLX loaded sample at different processing methods using deconvolution of XRD data. Data recorded from day 0 samples.

FIG. 5 is a bar graph showing average mesophase content of MLX loaded PCL samples at day 0. Mesophase content across different drug loadings were averaged for each processing method. Standard deviation indicates mesophase content variation between the different MLX loaded samples.

FIG. 6A shows panels of bar graphs depicting morphological content of 10% w/w MLX loaded PCL over 90 days. Samples were processed at 25° C. and 50° C. and analyzed by deconvolution for crystalline, amorphous, and mesomorphic content. Error bars indicate the standard deviation between morphological content across three different locations scanned by XRD on the sample. FIG. 6B shows panels of bar graphs showing morphological content of blank PCL samples over 90 days. Samples were processed at 25° C. and 50° C. and analyzed by deconvolution for crystalline, amorphous, and mesomorphic content. Error bars indicate the standard deviation between morphological content across three different locations scanned by XRD on the sample.

FIGS. 7A-7C are point graphs showing the effect of processing method on surface area normalized MLX release over 90 days. The normalized release of MLX was organized by processing method for (FIG. 7A) 5% w/w MLX loaded samples, (FIG. 7B) 10% w/w MLX loaded samples, and (FIG. 7C) 20% w/w MLX loaded samples. Error bars indicate standard deviation between triplicate samples for each condition.

FIGS. 8A-8C are point graphs showing the effect of processing method on cumulative MLX release over 90 days. Cumulative release of MLX was organized by drug loading for (FIG. 8A) first press samples, (FIG. 8B) 25° C. processed samples, and (FIG. 8C) 50° C. processed samples. Error bars indicate standard deviation between triplicate samples for each condition.

FIGS. 9A-9C are line graphs showing XRD profiles of 5% w/w, 10% w/w and 20% w/w dexamethasone (DEX) loaded PCL tablets with area normalized.

FIGS. 10A-10D are bar graphs of DEX loading PCL showing percent crystalline (FIG. 10A), amorphous (FIG. 10B), and mesophase (FIG. 10C) content calculated through XRD peak deconvolution. FIG. 10D shows mesophase content calculated through XRD peak deconvolution averaged across 5% w/w, 10% w/w, and 20% w/w loading.

FIGS. 11A-11C are point graphs showing cumulative drug release of 5% w/w, 10% w/w, and 20% w/w DEX loaded tablets over a span of 90 days (n=3).

FIGS. 12A-12C are line graphs showing diffusion flux as a function of time for 5% w/w, 10% w/w, and 20% w/w DEX loaded PCL tablets (n=3).

FIGS. 13A-13C are point graphs showing normalized drug release per surface area for 5% w/w, 10% w/w, and 20% w/w DEX loaded PCL tablets over a span of 3 months (n=3).

FIG. 14 is a line graphs showing diffusion flux of DEX calculated by assuming constant release rates.

FIGS. 15A-15C are line graphs showing the effect of processing conditions and increase in loading on the diffusion flux for (FIG. 15A) week 1, (FIG. 15B) week 2-7, and (FIG. 15C) week 8-26 (n=3).

FIG. 16 is a bar graph showing diffusion coefficients calculated using the Higuchi equation (n=3).

FIG. 17 is a point graphs showing a correlation between the calculated diffusion coefficient and mesophase content.

FIGS. 18A-18D are line graphs showing area normalized XRD diffraction patterns at different processing conditions (FIG. 18A) Blank PCL, (FIG. 18B) 5% w/w dolutegravir loading PCL tablets, (FIG. 18C) 10% w/w dolutegravir loading PCL tablets, and (FIG. 18D) 20% w/w dolutegravir loading PCL tablets.

FIGS. 19A-19C are bar graphs showing phase content of blank PCL tablets using the deconvolution method at day 0 before immersion into the release medium for the different manufacturing conditions (film cast, first press, processed 25° C. and processed 50° C.). (FIG. 19A) Percent crystallinity after each manufacturing step, (FIG. 19B) percent mesophase, (FIG. 19C) percent amorphous.

FIG. 20 is a point graph showing crystalline phase content as a function of percent of drug loading in PCL tablets for the four manufacturing steps: film cast (square), first press (circle), processed 25° C. (triangle) and processed 50° C. (diamond).

FIGS. 21A-21F are phase content of 10% Dolutegravir loaded PCL tablets using the deconvolution method at different time points (24 h, 72 h, 1 week, 2 weeks, 3 weeks, 1 month, 2 months, 3 months) with the Fityk software. (FIG. 21A) Change in crystallinity content of PCL for processed 25° C. tablets, (FIG. 21B) Change in mesophase content of PCL for processed 25° C. tablets, (FIG. 21C) Change in amorphous content of PCL for processed 25° C. tablets, (FIG. 21D) Change in crystallinity content of PCL for processed 50° C. tablets, (FIG. 21E) Change in mesophase content of PCL for processed 50° C. tablets, (FIG. 21F) Change in amorphous content of PCL for processed 50° C. tablets.

FIGS. 22A-22C are point graphs showing cumulative percent release per surface area (%/cm²) profiles of dolutegravir loaded PCL tablets. (FIG. 22A) 5% w/w dolutegravir loading, (FIG. 22B) 10% w/w dolutegravir loading, (FIG. 22C) 20% w/w dolutegravir loading. Each figure shows the first press tablets (square curves) which were hand pressed at 10,000 lbf in a tablet die at room temperature, and the hydraulically pressed tablets were processed at 10,000 lbf at both 25° C. (triangle curves) and 50° C. (circle curves).

FIG. 23 is a bar graph showing calculated diffusion coefficients using the Higuchi equation for first press, processed 25° C., processed 50° C. tablets with 5% w/w, 10% w/w, and 20% w/w DTG loading.

FIG. 24 is a point graph showing correlation between diffusion coefficient calculated by the Higuchi equation and the mesophase content estimated from XRD deconvolution from day 0 XRD scans. Squares: 5% w/w DTG, Circles: 10% w/w DTG and Triangles: 20% w/w DTG.

FIG. 25 is a bar graph comparing the diffusion coefficients of different hydrophobic drugs loaded in PCL tablets, both with and without mesophase. Briefly, PCL tablets were loaded with 10% (w/w) dolutgravir, dexamethasone, or meloxicam via a two-step film-cast/melt-cast process. The resulting tablets were used as a ‘without mesophase’ control in an in-vitro release experiment. Identical tablets containing each drug were further processed under 20,000 lbs at 50° C. to induce the PCL mesophase morphology. The resulting tablets were used to represent ‘with mesophase’ samples in an in vitro release experiment. The solid black bars represent the in-vitro diffusion coefficient calculated by the Higuchi equation for the ‘without mesophase’ control, while the lined bars represent the diffusion coefficient calculated for the ‘with mesophase’ samples.

FIG. 26 depicts the in-vitro release curve of DTG from both melt cast (without mesophase) and processed (with mesophase) PCL tablets.

FIG. 27 depicts the surface area normalized in-vitro release curve of 10% DTG loaded samples from both melt cast (without mesophase) and processed (with mesophase) PCL tablets.

FIG. 28 depicts the in-vitro release curve of 10% loaded dexamethasone from both melt cast (without mesophase) and processed (with mesophase) PCL tablets.

FIG. 29 depicts the surface area normalized in-vitro release curve of 10% loaded dexamethasone from both melt cast (without mesophase) and processed (with mesophase) PCL tablets.

FIG. 30 depicts the in-vitro release curve of 10% loaded meloxicam from both melt cast (without mesophase) and processed (with mesophase) PCL tablets.

FIG. 31 depicts the surface area normalized in-vitro release curve of 10% meloxicam from both melt cast (without mesophase) and processed (with mesophase) PCL tablets.

FIG. 32A shows the integrated XRD profiles of 100% PCL Film cast and processed at 10,000 lbs for 15 minutes at temperatures of no heat (First press), 25° C., and 50° C. Peak broadening indicates mesophase formation. FIG. 32B shows the integrated XRD profiles of 1% curcumin (CUR) 99 PCL Film cast and processed at 10,000 lbs, for 15 minutes at temperatures of no heat (First press), 25° C., and 50° C. Peak broadening indicates mesophase formation. FIG. 32C shows the integrated XRD profiles of 10% CUR 90% PCL Film cast and processed at 10,000 lbs, for 15 minutes at temperatures of no heat (First press), 25° C., and 50° C. Peak broadening indicates mesophase formation. FIG. 32D shows the integrated XRD profiles of 50% CUR 50% PCL Film cast and processed at 10,000 lbs, for 15 minutes at temperatures of no heat (First press), 25° C., and 50° C. Peak broadening indicates mesophase formation.

FIGS. 33A-33D show the XRD scattering profiles of PCL systems loaded with 1% curcumin (CUR) and analyzed by deconvoluting the peaks. Peak broadening indicates mesophase formation.

FIGS. 34A-34D are bar graphs showing the calculation of percent crystalline, amorphous, and mesophase content through XRD. Shown are 100% PCL (FIG. 34A), 1% CUR 99% PCL (FIG. 34B), 10% CUR 90% PCL (FIG. 34C), and 50% CUR 50% PCL (FIG. 34D).

FIGS. 35A-35D are bar graphs showing DSC Delta H and delta H corrected calculation for different drug loading and different process conditions to analyze the interaction between the drug, polymer, and recrystallization characteristics for film cast (FIG. 35A), first press (FIG. 35B), 25° C. press (FIG. 35C), and 50° C. press (FIG. 35D).

FIGS. 36A and 36B are graphs showing X-Ray diffraction curve of segment before (FIG. 36A) and after stretching (FIG. 36B) indicating a change in intensity and broadening indication mesophase formation.

FIG. 37 is a collection of XRD and 2D-XRD captures of two arbitrary sites on hand pressed 100% PLA film casted samples under 5,000 lbs. of pressure for 10 minutes (see a₁, a₂, b₁, b₂), 10,000 lbs. of pressure for 5 minutes (see c₁, c₂, d₁, d₂) and 10 minutes (e₁, e₂, f₁, f₂); 15,000 lbs. of pressure for 5 minutes (g₁, g₂, h₁, h₂), 10 minutes (i₁, i₂, j₁, j₂) and 15 minutes (k₁, k₂, l₁, l₂).

FIG. 38 is a collection of XRD plots and 2D-XRD captures of hand press of 1% Cur 99% PLA under 5000 lbs. of pressure for 5 minutes (a₁, a₂) and 10 minutes (b₁, b₂); 10000 lbs. of pressure for 5 minutes (c₁, c₂) and 10 minutes (d₁, d₂); 15000 lbs. of pressure for 5 minutes (e₁, e₂); 10 minutes (f₁, f₂), and 15 minutes (g₁, g₂).

FIG. 39 is a line graph showing cumulative release (%) of 1%, 5%, 30% and 50% dolutegravir loaded tablets, which was obtained using HPLC for 6 weeks (42 days).

### DETAILED DESCRIPTION OF THE INVENTION

### I. Definitions

“About,” as relates to numerical values described herein, refers to a value that is ±10% of the specified value. Specific values within this range are ±1%, ±2%, ±3%, ±4%, ±5%, ±6%, ±7%, ±8%, ±9%, and ±10%.

The term “hydrophobic,” as used herein, refers to the property of lacking affinity for or repelling water. For example, the more hydrophobic a drug, the more that drug tends to not dissolve in, not mix with, or not be wetted by water. Hydrophobicity can be quantified by measuring a drug's partition coefficient between water (or a buffered aqueous solution) and a water-immiscible organic solvent, such as octanol, methylene chloride, or methyl tert-butyl ether. If after equilibration a greater concentration of the drug is attained in the organic solvent than in water, the drug is considered hydrophobic. For example, if the organic solvent is octanol, then a positive log P value indicates that the drug is hydrophobic. “Hydrophobic” may also refer to a polymer or drug that when applied to a surface, such as glass, forms a contact angle with water, which is greater than the contact angle of water on a surface of glass without the polymer or drug.

“Small molecule” generally refers to an organic molecule that is less than about 2500 Da, such as between 100 Da and 2500 Da. Typically, small molecules are non-polymeric and/or non-oligomeric.

### II. Compositions

Described are compositions for controlled release, preferably long-term controlled release, of hydrophobic drugs in subjects in need of these drugs. One of the main aims of innovation in the field of long-term drug delivery is to lessen the variations in drug uptake, so that the desired drug dosage stays in a subject's system for a longer time. The compositions contain a polymer matrix containing a hydrophobic polymer and a hydrophobic drug dispersed within the polymer matrix. The mesophase can be formed from a single polymer or from a blend of two or more hydrophobic polymers, preferably including at least a polyester, such as a linear aliphatic polyester. Examples of blends include poly(lactic acid) and poly(caprolactone), poly(lactic acid) and poly(lactic-co-glycolic acid), or poly(caprolactone) and poly(lactic-co-glycolic acid).

In some forms, the polymer matrix further contains a biocompatible polymer having a non-biodegradable backbone, or preferably a biocompatible, non-biodegradable polymer. Examples of these polymers include polyvinyl alcohol; polyalkylene oxides (e.g., poly(ethylene glycol), poly(ethylene oxide), poly(propylene oxide), and copolymers thereof, such as poloxamers); polymethacrylates; and copolymers of these polymers. Polymethacrylates are described in dos Santos, et al., Pharmaceutics 2021, 13 (9), 1424, the contents of which are herein incorporated by reference.

In some forms, excipients can be included in the polymer matrix. The excipients can be plasticizers, stabilizers or drug release-altering compounds. In these forms, the excipients can constitute between about 1% and about 20%, between about 1% and about 15%, between about 1% and about 10%, between about 1% and about 5% of the composition.

The polymer matrix has a mesophase content between about 5% and about 50% (such as between about 5% and about 45%, about 5% and about 40%, about 5% and about 35%, and between about 5% and about 30%) as measured using X-ray diffraction techniques at atmospheric pressure and room temperature (between 20° C. and 30° C.) or as measured using differential scanning calorimetry at a range of temperature below the melting temperature of the hydrophobic polymer. The mesophase confers a certain degree of order and flexibility to the polymer matrix by providing positional order, as well as conformational and orientational disorder to control the diffusion of drugs within the polymeric matrix.

The disclosed compositions also encompass delivery systems that can be processed to induce a mesophase. Some of these delivery systems include, but are not limited to, film and melt cast, cylinders rods, tubes microspheres and microcapsules, the mesophase can be induce after the delivery is already made by just applying pressure.

### A. Hydrophobic and Amphiphilic Polymers

Preferred polymers to include in the polymer matrix are hydrophobic polymers. In some forms, the hydrophobic polymer is a biodegradable polymer, preferably a biodegradable thermoplastic polymer. In some forms, the hydrophobic polymer is selected from a polyester, polyanhydride, poly(ortho) esters, poly(p-dioxanone), poly(polyurethane), polycarbonate, and a combination thereof. In some forms, the hydrophobic polymer is a polyester, preferably a linear aliphatic polyester. In some forms, the hydrophobic polymer is selected from the group consisting of a poly(lactic acid), poly(caprolactone), poly(lactic acid-co-glycolic acid), poly(pentadecalactone), poly(hydroxybutyrate-co-hydroxyvalerate), poly(hydroxybutyrate), polybutylene succinate, and a combination thereof.

In some forms, the polymer matrix is formed from one class of these hydrophobic polymers. In other forms, blends of different classes of these hydrophobic polymers can be employed to form the polymer matrix. Preferably, the blends include at least a polyester, such as a such as a linear aliphatic polyester. Examples of blends include poly(lactic acid) and poly(caprolactone), poly(lactic acid) and poly(lactic-co-glycolic acid), or poly(caprolactone) and poly(lactic-co-glycolic acid).

In some forms, the polymer matrix contains an amphiphilic polymer containing a hydrophobic segment (preferably a hydrophobic polymer segment) and a hydrophilic segment (preferably a hydrophilic polymer segment). Preferably, the hydrophobic segment contains one or more of the hydrophobic polymers described herein. Preferably, the hydrophilic segment contains a hydrophilic thermoplastic polymer. Examples of hydrophilic polymers that can be included in the hydrophilic segment include, but are not limited to, poly(ethylene oxide), poly(ethylene glycol), poly(vinyl alcohol), poly(aminoethylmethacrylate), poly(hydroxy ethylmethacrylate), poly(acrylonitrile), or poly(amide).

In some forms, the polymers are biocompatible non-bioerodible hydrophobic polymers (e.g., homopolymers, copolymers, or blends thereof). In some forms, these polymers include, but are not limited to, poly(acrylates), poly(methacrylates), polycarbonates, polypropylenes, polyalkylenes, hydrophobic polyalkylene glycols, hydrophobic polyalkylene oxides, poly(alkylene terephthalates), poly(vinyl ethers), poly(vinyl halides), polysiloxanes, polyurethanes, hydroxyalkyl celluloses, cellulose ethers, nitro celluloses, methyl cellulose, ethyl cellulose, cellulose acetate, cellulose propionate, cellulose acetate butyrate, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenylmethacrylate), poly(methyl acrylate), poly(isopropyl acrylate), poly(isobutylacrylate), poly(octadecyl acrylate), polyethylene, poly(ethylene terephthalatepoly(vinyl acetate), and poly vinyl chloride polystyrene, polyethylene, copolymers and blends thereof.

### B. Hydrophobic Drugs

The compositions contain one or more hydrophobic drugs. The one or more hydrophobic drug(s) are distributed within the polymer matrix. Preferably, the one or more hydrophobic drugs are homogeneously distributed within the polymer matrix. Preferably, the one or more hydrophobic drugs are not covalently bound to a polymer (e.g., a hydrophobic polymer) that forms the polymeric matrix, such that release of the drug is primarily through diffusion from the matrix without the requirement of the cleavage of a covalent bond to release the drug from the matrix. In some forms, the one or more hydrophobic drugs can also affect mesophase formation, such as enhance formation of a mesophase in the polymer matrix.

In some forms, the one or more hydrophobic drugs have a loading between about 1% w/w and about 60% w/w, about 2.5% w/w and about 60% w/w, about 5% w/w and about 60% w/w, about 1% w/w and about 50% w/w, about 2.5% w/w and about 50% w/w, about 5% w/w and about 50% w/w, 1% w/w and about 25% w/w, of the polymeric matrix. In some forms, the one or more hydrophobic drugs have a loading between about 1% w/w and about 60% w/w, about 1% w/w and about 50% w/w, or about 1% w/w and about 25% w/w, of the composition. The percent weight of the drug referenced herein refers to the total percent weight of either a single hydrophobic drug or two or more hydrophobic drugs. The percent weight of the hydrophobic drug refers to the weight of the hydrophobic drug compared to the total weight of the drug and polymer used to form the polymeric matrix and optional excipients. Preferably, the precent weight of the drug is determined from the amount of the drug used to form the composition, the polymer used to form the polymeric matrix, and other optional excipients, given that the composition is preferably formed via one or more physical processes (e.g., mixing, compressing, heating, and/or cooling). In some forms, the compositions have a higher proportion of the hydrophobic drugs dispersed within the polymer matrix than on the surface of the polymer matrix. In some forms, the hydrophobic drugs are dispersed within the polymer matrix and are not on the surface of the polymer matrix.

In some forms, the hydrophobic drug is released from the composition for at least one day, such as such as such as such as between one day and a year, between one and 200 days, inclusive, such as between one and 180 days, inclusive, such as between one and 150 days, inclusive, between one and 100 days, inclusive, between one and 75 days, inclusive, or between one and 50 days, inclusive.

Examples of hydrophobic classes of drugs include, but are not limited to, steroids, hydrophobic anti-viral agents, hydrophobic anti-inflammatory agents, hydrophobic anti-fungal agents, hydrophobic hormones, hydrophobic chemotherapeutic agents, hydrophobic antibiotics, hydrophobic analgesics, etc. Some examples of hydrophobic drugs include dexamethasone, meloxicam, dolutegravir, docetaxel, paclitaxel, doxorubicin, tamoxifen, camptothecins, methotrexate, curcumin, etc.

### C. Excipients

In some forms, excipients can be included in the polymer matrix. Preferably, these excipients are included before induction of the mesophase. The excipients can be plasticizers, stabilizers, or drug release-altering compounds. In these forms, the excipients can constitute between about 1% and about 20%, between about 1% and about 15%, between about 1% and about 10%, between about 1% and about 5% of the composition, such as 1%, 2%, 3%, 4%, 5%, 6%, 7%, 8%, 9%, 10%, 11%, 12%, 13%, 14%, 15%, 16%, 17%, 18%, 19%, or 20%. Plasticizers may be used to help induce more mesophase (i.e., acting as a ‘lubricant’ for polymer chain mobility and facilitating mesophase formation). Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. “Stabilizers” may be used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA). The drug release-altering excipients can be used to tune the rate of drug release through the mesophase in the polymer matrix. For instance, hydrophobic excipients slow drug release, while hydrophilic excipients speed up release.

### II. Methods of Making the Disclosed Compositions

Described herein are processes suitable for inducing a mesophase: applying pressure, applying heat and pressure, or applying stress (e.g., stretching a polymeric matrix). Also provided, are specific methods for inducing mesophase formation in compositions containing a polymer matrix containing (a) a hydrophobic polymer and/or an amphiphilic polymer and (b) a hydrophobic drug.

The initial step involves mixing the drug (preferably hydrophobic drug) and the polymer (such as hydrophobic polymer and/or an amphiphilic polymer): this can be achieved by simple physical mixing of solid composition and then applying the additional steps described below, or by dissolving the drug and polymer in solvents that are good solvents for both polymer and drug, casting a film, evaporating the solvent, and then processing the composition.

The methods for inducing the mesophase include the steps of step (i) heating a combination of the hydrophobic polymer and/or amphiphilic polymer, and the hydrophobic drug to a first temperature (such as, between 22° C. and 250° C., e.g., 25° C. and 250° C., 22° C. and 120° C., 22° C., 40° C., 50° C., 63° C., 70° C., 85° C., and 120° C.), depending on the type of polymer used, step (ii) applying to the combination (e.g., a mixture) an elevated pressure greater than atmospheric pressure and at the first temperature, and step (iii) cooling the combination to a desired temperature, while the pressure is still applied. The temperature involved, as noted above, will depend on the type of polymer, such that the mesophase can be induced below the glass transition temperature of the polymer and up to below the melting temperature of the polymer. In some forms, the heating in step (i) is performed in a mold at a suitable temperature to enhance fusion of the composition, i.e., melt casting the combination. In some forms, the methods further involve applying to the combination a first pressure prior to step (i).

In some forms, the methods further include the step of assessing the mesophase content at a second temperature below the first temperature, using X-ray diffraction techniques at atmospheric pressure, after step (iii) referenced above.

Preferably, the first pressure is greater than about 13 MPa, such as between about 14 MPa and about 275 MPa, between about 50 MPa and about 275 MPa, between about 14 MPa and about 140 MPa, between about 30 MPa and about 140 MPa, or between about 50 MPa and 140 MPa, such as about 65 MPa, about 66 MPa, about 67 MPa, about 68 MPa, about 69 MPa, or about 70 MPa. Preferably, the first temperature referenced above is a temperature below the melting temperature of the hydrophobic polymer or amphiphilic polymer. Preferably, the second temperature is room temperature (between 20° C. and 30° C.).

In some forms, step (ii) is performed within a time between about 1 min and about 20 mins, such as between about 5 mins and about 15 mins. In some forms, step (iii) is performed while maintaining the combination at the elevated pressure attained in step (ii). Preferably, the elevated pressure is greater than about 13 MPa, such as between about 14 MPa and about 275 MPa, between about 50 MPa and about 275 MPa, between about 14 MPa and about 140 MPa, or between about 30 MPa and about 140 MPa, such as about 125 MPa, about 130 MPa, about 135 MPa, about 138 MPa, or about 140 MPa. In some specific forms, the first pressure is about 69 MPa, and the elevated pressure is about 138 MPa.

Alternatively, the methods for inducing the mesophase involve film casting a composition containing the hydrophobic polymer and/or amphiphilic polymer, and the hydrophobic drug, and then applying stress to the film, such as stretching the film until it forms a mesophase. The existence of a mesophase can be confirmed using X-ray.

Preferably, upon completion of these steps, the polymer matrix has a mesophase content between about 5% and about 50% (such as between about 5% and about 45%, about 5% and about 40%, about 5% and about 35%, and between about 5% and about 30%) as measured using X-ray diffraction techniques at atmospheric pressure.

Suitable polymers for use in the described methods include hydrophobic polymers, preferably those that are biodegradable polymers, more preferably biodegradable thermoplastic polymers. The hydrophobic polymers can be selected from a polyester, polyanhydride, poly(ortho) esters, poly(p-dioxanone), poly(polyurethane), polycarbonate, and a combination thereof. In some forms, the hydrophobic polymer is a polyester, preferably a linear aliphatic polyester. In some forms, the hydrophobic polymer is selected from the group consisting of a poly(lactic acid), poly(caprolactone), poly(lactic acid-co-glycolic acid), poly(pentadecalactone), poly(hydroxybutyrate-co-hydroxyvalerate), poly(hydroxybutyrate), polybutylene succinate, and a combination thereof. An amphiphilic polymer containing a hydrophobic segment (preferably a hydrophobic polymer segment) and a hydrophilic segment (preferably a hydrophilic polymer segment) can also be used to form the polymer matrix. Preferably, the hydrophobic segment contains one or more of the hydrophobic polymers described herein. Preferably, the hydrophilic segment contains a hydrophilic thermoplastic polymer. Examples of hydrophilic polymers that can be included in the hydrophilic segment include, but are not limited to, poly(ethylene oxide), poly(ethylene glycol), poly(vinyl alcohol), poly(aminoethylmethacrylate), poly(hydroxy ethylmethacrylate), poly(acrylonitrile), or poly(amide). Additional polymers that can be included in the polymer matrix include a biocompatible polymer having a non-biodegradable backbone, or preferably a biocompatible, non-biodegradable polymer. Examples of these polymers include polyvinyl alcohol; polyalkylene oxides (e.g., poly(ethylene glycol), poly(ethylene oxide), poly(propylene oxide), and copolymers thereof, such as poloxamers); polymethacrylates; and copolymers of these polymers.

In some forms, the polymer matrix contains a blend of (i) different classes of these hydrophobic polymers, (ii) different amphiphilic polymers, (iii) a hydrophobic polymer and an amphiphilic polymer, or (iv) a biocompatible polymer having a non-biodegradable backbone, or preferably a biocompatible, non-biodegradable polymer, and one or more of hydrophobic polymers, and amphiphilic polymers. Preferably, the blends include at least a polyester, such as a such as a linear aliphatic polyester. Examples of blends include poly(lactic acid) and poly(caprolactone), poly(lactic acid) and poly(lactic-co-glycolic acid), or poly(caprolactone) and poly(lactic-co-glycolic acid).

Before applying pressure or stress in each case, preferably, there is an initial preparation step that involves mixing the polymer and the drug together. Mixing the polymer and the drug can depend on their physical properties. If both the polymer and the drug are solid particles, simple physical mixing can be done. Alternatively, both the drug and polymer can be mixed and dissolved in a solvent that is suitable for both, followed by film casting and solvent evaporation. Another method involves dissolving the polymer in a suitable solvent and then dispersing the drug. Lastly, the polymer and drug can be mixed, melted, and then extruded together. In this final method, care is taken not to damage the drug. In sum, one can form any delivery system and then apply the pressure. In the case where the delivery systems are microparticles, for example, pressure can be applied after formation of the microparticles in a pressure vessel. In such an instance, appropriate measures are taken to prevent compromising the shape and/or size of the microparticles. The drug and polymer must be mixed before applying the steps described to induce the mesophase.

### III. Methods of Using the Disclosed Compositions

The compositions described herein, can be used as a versatile platform for delivery of one or more hydrophobic drugs, preferably long-term delivery. Given their demonstrated long-term release kinetics, the disclosed compositions can find application in chronic diseases or disorders that require patients to receive multiple doses of one or more drugs. The compositions can be used to deliver an effective amount of one or more of these hydrophobic drugs. The amount of agent to be administered can be readily determined by the prescribing physician and is dependent on the age and weight of the patient and the disease or disorder to be treated.

Methods of administration of the compositions can be enterally or parenterally. Enteral administration includes oral administration i.e., administration to or by way of the mouth, to provide uptake through the GI tract. Parenteral administration includes the following routes of administration: buccal, by placement in the buccal cavity for uptake through the mouth; mucosal, e.g., intranasally, or intravaginal administration, or direct application to a mucous membrane in the subject; intracranial; subcutaneous; intramuscular; intraperitoneal; transdermal, etc. Preferred methods of administration involve implantation.

The compositions can be administered as capsules, tablets, troches, implants (such as films, membranes, fibers, or cylinders), etc. The films, membranes, fibers, or cylinders can be single-layered, multi-layered, unidirectional (i.e., release drug through only side), multi-directional (i.e., release drug through at least two sides), or a combination thereof. Preferably, the compositions are administered in a solid form.

The disclosed systems and methods of use can be further understood through the following enumerated paragraphs or embodiments.

1. A composition containing a polymer matrix containing a (i) hydrophobic polymer and/or an amphiphilic polymer and (ii) a hydrophobic drug, wherein the polymer matrix has a mesophase content between about 5% and about 50% (such as between about 5% and about 45%, about 5% and about 40%, about 5% and about 35%, and between about 5% and about 30%) as measured using X-ray diffraction techniques at atmospheric pressure and room temperature (between 20° C. and 30° C.) or differential scanning calorimetry at a range of temperatures below the melting temperature of the hydrophobic polymer or amphiphilic polymer,
wherein the amphiphilic polymer contains a hydrophobic polymer segment and a hydrophilic polymer segment.

2. The composition of paragraph 1, wherein the hydrophobic polymer and/or the hydrophobic polymer segment is a biodegradable polymer, preferably a biodegradable thermoplastic polymer.

3. The composition of paragraph 1 or 2, wherein the hydrophobic polymer and/or hydrophobic polymer segment are selected from the group composed of a polyester, polyanhydride, poly(ortho) esters, poly(p-dioxanone), poly(polyurethane), polycarbonate, and a combination thereof.

4. The composition of any one of paragraphs 1 to 3, wherein the hydrophobic polymer and/or hydrophobic polymer segment is a polyester, preferably a linear aliphatic polyester.

5. The composition of any one of paragraphs 1 to 4, wherein the hydrophobic polymer and/or hydrophobic polymer segment is selected from the group consisting of a poly(lactic acid), poly(caprolactone), poly(lactic acid-co-glycolic acid), poly(pentadecalactone), poly(hydroxybutyrate-co-hydroxyvalerate), poly(hydroxybutyrate), polybutylene succinate, and a combination thereof.

6. The composition of any one of paragraphs 1 to 5, wherein the hydrophilic polymer segment contains poly(ethylene oxide), poly(ethylene glycol), poly(vinyl alcohol), poly(aminoethylmethacrylate), poly(hydroxy ethylmethacrylate), poly(acrylonitrile), or poly(amide).

7. The composition of any one of paragraphs 1 to 6, wherein the hydrophobic drug is homogeneously distributed within the polymer matrix.

8 The composition of any one of paragraphs 1 to 7, wherein the hydrophobic drug is not covalently bound to the hydrophobic polymer.

9. The composition of any one of paragraphs 1 to 8, wherein the hydrophobic drug has a loading between about 1% w/w and about 50% w/w, about 2.5% w/w and about 50% w/w, about 5% w/w and about 50% w/w, about 1% w/w and about 35% w/w, between about 2.5% w/w and about 35% w/w, between about 5% w/w and about 35% w/w, between about 5% w/w and about 30% w/w, or between about 5% w/w and about 25% w/w.

10. The composition of any one of paragraphs 1 to 9, wherein the hydrophobic drug is released from the composition at least one day, such as such as between one and one year, inclusive, one and 200 days, inclusive, such as between one and 180 days, inclusive, such as between one and 150 days, inclusive, between one and 100 days, inclusive, between one and 75 days, inclusive, or between one and 50 days, inclusive.

11. The composition of any one of paragraphs 1 to 10, wherein the polymer matrix is formed by a method involving:
(i) exposing a combination (e.g., a mixture) of the hydrophobic polymer and the hydrophobic drug to a first temperature and a pressure greater than atmospheric pressure.

12. The composition of paragraph 11, wherein the first temperature is a temperature less than the melting temperature of the polymer.

13. The composition of paragraph 11 or 12, wherein prior to step (i), the combination is heated to the first temperature.

14. The composition of any one of paragraphs 11 to 13, wherein after step (i), the mixture is cooled while maintaining the pressure attained at step (i).

15. The composition of any one of paragraphs 1 to 14, wherein the composition is in the form of capsules, tablets, troches, or implants (such as films, membranes, fibers, or cylinders), etc.

16. The composition of any one of paragraph 1 to 15, wherein the composition is in a solid form.

17. The composition of any one of paragraphs 1 to 16, further containing an excipient dispersed within or associated with the polymer matrix, preferably wherein the excipient is a plasticizer, stabilizer, or drug-release altering agent.

18. A method of administering a hydrophobic drug to a subject in need thereof, the method involving implanting or injecting into the subject the composition of any one of paragraphs 1 to 17.

19. A method of inducing a mesophase content in a polymeric matrix containing a hydrophobic polymer and/or amphiphilic polymer, and a hydrophobic drug, the method involving:
(A)
   (i) heating a combination of the hydrophobic polymer and/or amphiphilic polymer, and the hydrophobic drug to a first temperature (such as, between 22° C. and 120° C., e.g., 22° C., 40° C., 50° C., 63° C., 70° C., 85° C., and 120° C.),
   (ii) applying to the combination (e.g., a mixture) an elevated pressure greater than atmospheric pressure and at the first temperature, and
   (iii) cooling the combination to a desired temperature, or
(B)
   (bi) film casting a combination of the hydrophobic polymer and/or amphiphilic polymer, and the hydrophobic drug to form a film, and
   (bii) stretching the film until it reaches its breaking point,
wherein the amphiphilic polymer contains a hydrophobic polymer segment and a hydrophilic polymer segment.

20. The method of paragraph 19, involving applying to the combination a first pressure prior to step (i).

21. The method of paragraph 20, wherein the first pressure is greater than about 13 MPa, such as between about 14 MPa and about 275 MPa, between about 50 MPa and about 275 MPa, between about 14 MPa and about 140 MPa, between about 30 MPa and about 140 MPa, or between about 50 MPa and 140 MPa, such as about 65 MPa, about 66 MPa, about 67 MPa, about 68 MPa, about 69 MPa, or about 70 MPa.

22. The method of any one of paragraphs 19 to 21, wherein the first temperature is a temperature below the melting temperature of the hydrophobic polymer and/or the amphiphilic polymer.

23. The method of any one of paragraphs 19 to 22, further involving step (iv) assessing the mesophase content at a second temperature below the first temperature, using X-ray diffraction techniques at atmospheric pressure.

24. The method of paragraph 23, wherein the second temperature is room temperature (between 20° C. and 30° C.).

25. The method of any one of paragraphs 19 to 24, wherein step (ii) is performed within a time between about 1 min and about 20 mins, such as between about 5 mins and about 15 mins.

26. The method of any one of paragraphs 19 to 25, wherein step (iii) is performed while maintaining the combination at the elevated pressure.

27. The method of any one of paragraphs 19 to 26, wherein the elevated pressure is greater than about 13 MPa, such as between about 14 MPa and about 275 MPa, between about 50 MPa and about 275 MPa, between about 14 MPa and about 140 MPa, or between about 30 MPa and about 140 MPa, such as about 125 MPa, about 130 MPa, about 135 MPa, about 138 MPa, or about 140 MPa.

28. The method of any one of paragraphs 19 to 27, wherein after step (iii), the polymer matrix has a mesophase content between about 5% and about 50% (such as between about 5% and about 45%, about 5% and about 40%, about 5% and about 35%, and between about 5% and about 30%) as measured using X-ray diffraction techniques at atmospheric pressure.

29. The method of any one of paragraphs 19 to 28, wherein hydrophobic polymer and/or hydrophobic polymer segment contains a biodegradable polymer, preferably a biodegradable thermoplastic polymer.

30. The method of any one of paragraphs 19 to 29, wherein the hydrophobic polymer and/or hydrophobic polymer segment is selected from the group composed of a polyester, polyanhydride, poly(ortho) esters, poly(p-dioxanone), poly(polyurethane), polycarbonate, and a combination thereof.

31. The method of any one of paragraphs 19 to 30, wherein the hydrophobic polymer and/or hydrophobic polymer segment is a polyester, preferably a linear aliphatic polyester.

32. The method of any one of paragraphs 19 to 31, wherein the hydrophobic polymer and/or hydrophobic polymer segment is selected from the group composed of a poly(lactic acid), poly(caprolactone), poly(lactic acid-co-glycolic acid), poly(pentadecalactone), poly(hydroxybutyrate-co-hydroxyvalerate), poly(hydroxybutyrate), polybutylene succinate, and a combination thereof.

33. The method of any one of paragraphs 19 to 32, wherein the hydrophilic polymer segment contains poly(ethylene oxide), poly(ethylene glycol), poly(vinyl alcohol), poly(aminoethylmethacrylate), poly(hydroxy ethylmethacrylate), poly(acrylonitrile), or poly(amide).

The present invention will be further understood by reference to the following non-limiting examples.

### EXAMPLES

Much research has been conducted on the amorphous and semi-crystalline structures of polymers. However, mesophase induction of natural polymers remains a relatively unexplored area. Mesophases are polymeric structures that exist in-between the common amorphous and semi-crystalline structures. The three main mesophases that have been identified include the plastic crystal, condis crystal, and liquid crystal structures, (FIG. 1). The studies described herein utilize (i) (a) film casting, (b) temperature treatment, (c) pressure and temperature treatment, or (d) melt casting, temperature treatment, and pressure treatment to induce mesophase formation in polymers, particularly linear hydrophobic polymers, and (ii) a range of analytical methods to explore the drug release properties of these materials.

### Example 1. Induction of Mesophase in Polycaprolactone and its Effect on the Long-Term Delivery of Meloxicam

### Materials and Methods

i. Solvent Film Casting

The solvent film casting method was first used to create controls for the morphological studies as well as a tool to mix the drug and the polymer. Mixing the drug and polymer serves as the first step in the further processing procedures. CAPA 640 polycaprolactone (PCL) was received from Perstorp Holding AB (Perstorp, Sweden) for scientific use with a molecular weight (MW) of 37 kg/mol. Each film was produced with 250 mg of total theoretical material (i.e., drug plus polymer initially weighed out), whether it be only PCL or PCL/meloxicam (MLX) combinations.

For the blank PCL samples, a high-precision lab analytical balance scale (0.0001 g) was used to measure 250 mg of PCL (±0.1 mg) and added to a 20 mL scintillation vial. 2 mL of dichloromethane (DCM) was then added to the vial to solubilize the polymer. To fully solubilize the PCL in the DCM, a combination of sonication and vortexing was used and repeated 3 times equally spread out over a span of 15 minutes. Once fully solubilized, the solution was quickly pipetted using an electric pipette with a glass pipette tip into a 62 mm glass petri dish. The solution was evenly distributed around the petri dish and left undisturbed under a fume hood for 24 hours to allow the DCM to fully evaporate and leave a solid film of pure PCL. After 24 hours of evaporation, the film was retrieved using tweezers and stored separately in an appropriately sized plastic bag for later analysis and further processing. This procedure was repeated for all blank PCL films.

For the samples of PCL with 5% w/w, 10% w/w, and 20% w/w MLX loading, a similar procedure was used to produce the desired films. To keep the total theoretical material constant at 250 mg, MLX mass measurements were based on the 250 mg total theoretical mass. PCL to be used for the 5% w/w, 10% w/w, and 20% w/w MLX loaded formulations was measured to 237.5 mg, 225 mg, and 200 mg respectively (±0.1 mg) using a high-precision lab analytical balance scale (0.0001 g). 12.5 mg, 25 mg, and 50 mg of MLX was measured (±0.1 mg) for 5% w/w, 10% w/w, and 20% w/w MLX loaded samples respectively using a high-precision lab analytical balance scale (0.0001 g) into 20 mL scintillation vials. 1 mL of DCM was then added to each vial to initially solubilize the MLX. The respective amount of PCL was then added to each vial, followed by an additional 1 mL of DCM for a total amount of 2 mL of DCM. In an aim to fully solubilize both the drug and polymer in the DCM, the vials underwent sonication and vortexing 3 times equally spread out over a span of 15 minutes. The solution was then quickly pipetted using an electric pipette with a glass pipette tip into a 62 mm glass petri dish. The solution was evenly distributed around the petri dish and left undisturbed under a fume hood for 24 hours to allow the DCM to fully evaporate and leave a solid film of PCL and MLX. After 24 hours of evaporation, the films were retrieved using tweezers and stored separately in plastic bags for later analysis and/or further processing. This procedure was repeated to obtain additional films for further processing.

ii. First Press

Solvent casted films that were assigned for further processing underwent the first press procedure. The procedure consisted of folding up the films into a cylindrical die and pressing the samples at 10,000 lbs of pressure using a manual hand press. This pressing was kept constant at 10,000 lbs of pressure for 5 minutes for each sample to ensure a cylindrical tablet was formed. First press samples were measured for surface area to account for differences in shape compared to other processing methods. This procedure was repeated to obtain additional samples for further analysis and processing.

iii. Second Press

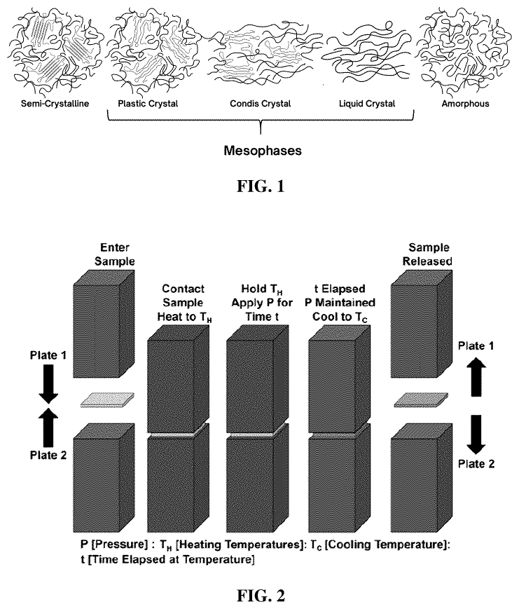

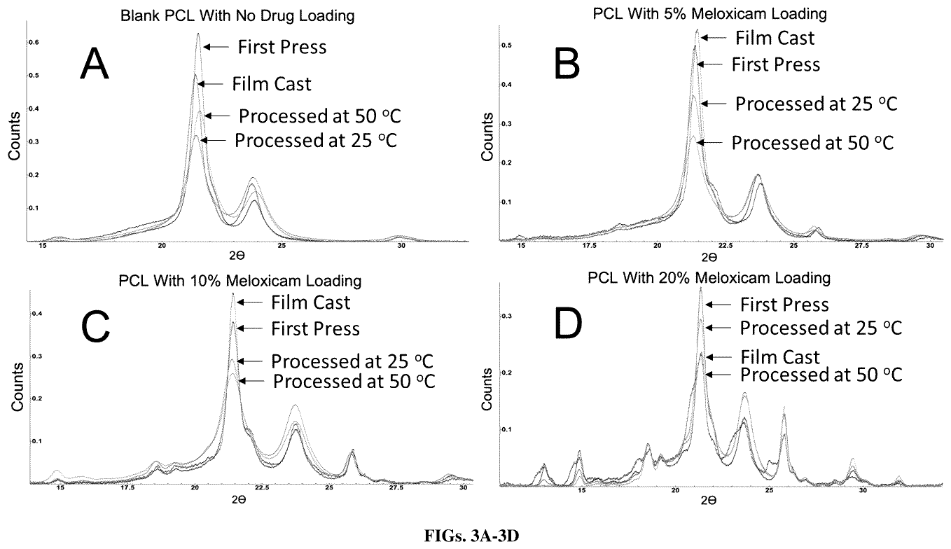

First press samples that were designated to be used as temperature-varied processed samples were pressed using a Carver Auto Series Hydraulic Press (Carver, Wabash, Indiana) equipped with a temperature control system. The hydraulic press includes an upper and lower 4″×4″ stainless steel plate that can be heated or cooled. The two plates were first set to heat to the desired temperature for the samples, either 25° C. or 50° C. The first press tablets were then placed on the bottom plate and the press was activated so that the bottom and top plates were both touching and applying heat to the first press tablets between the two plates. These samples were exposed to the desired temperature for 10 minutes. After 10 minutes, the press was activated to apply 10,000 lbs of pressure to the samples while still applying heat. This allowed the polymeric chains to flow in the samples while keeping the pressure constant in comparison to the first press samples. After 5 minutes of pressing, the applied temperature and pressure were reduced back to normal conditions, i.e., room temperature and atmospheric pressure, allowing the samples to cool and lock in their changed morphological state. Samples were cooled for 5 minutes during this period. Finally, the plates were separated (FIG. 2) and processed samples were collected for analysis (FIGS. 3A-8C). Second press samples were measured for surface area to account for differences in shape compared to other processing methods.

iv. Morphological Studies

About 60 samples were produced for this study. The general idea was to prepare samples under various conditions and expose them to aqueous conditions for 90 days and evaluate the morphology of the samples by X-ray. This will allow the determination of the stability of the mesophases at 37° C.

Samples processed at 25° C. and 50° C. were used to determine the change in polymer morphology over time when being subjected to an environment in which MLX could be eluted. This was done by placing each sample in separate falcon tubes along with 20 mL of phosphate buffered saline (PBS) with a pH of 7.4. PBS was used to mimic the sink conditions of a physiological environment. The falcon tubes were stored in an incubator at 37° C. until taken out for retrieval of solution at each timepoint. The time points that were used ranged from 1-90 days: 1 day, 3 days, 7 days, 14 days, 21 days, 30 days, 60 days, and 90 days. At a respective timepoint, the falcon tubes were gathered from the incubator and samples were retrieved from the PBS. The samples were then frozen and subjected to 48 hours of lyophilization before analysis.

v. X-Ray Diffraction and Analysis

The x-ray diffraction (XRD) system used was a Bruker D-8 Advance X-Ray Powder Diffraction system with a Cu X-Ray tube operating at 40 kV and 40 mA (1600 W) equipped with a Bruker Vantec-500 Xe—CO2 gas filled detector with a 13.5 cm diameter window set at 20 cm from the goniometer center. The system wavelength was 0.15418 nm and DaVinci Software was used to collect quantitative data from the diffraction frames. XRD was conducted on all the morphological samples to further determine the crystalline, amorphous, and mesomorphic content of each of the samples. Three different areas along each sample were analyzed using XRD to account for variation in morphological content within samples.

The quantitative XRD data collected from the XRD frames was analyzed using Fityk software, allowing for use of deconvolution fitting of gaussian curves to determine the area under the XRD curves. The software allows for multiple curves to be plotted for the different polymer contents, allowing for the determination of crystalline, amorphous, and mesophase content through the area found under the peaks.

vi. Calibration Curve

Calibration curves were created for MLX to best determine the release profile of MLX under sink conditions. MLX was dissolved in PBS and exhibited to a series of dilutions in which MLX solutions with concentrations of 250 μg/mL to 1 μg/mL were prepared. The diluted solutions were loaded onto a 96 well quartz plate and analyzed using a Spectra max 384 Plus Ultraviolet Spectrophotometer with SoftMax PRO 4.3.1 software to measure the absorbance of each solution. MLX is reported to have a maximum absorbance of 363 nm, so UV absorbance was measured at 361-365 nm to include measurements to find the true maximum absorbance for the samples used in the study (Bae, J.-W., Kim, M.-J., Jang, C.-G., & Lee, S.-Y. (2007). Determination of meloxicam in human plasma using a HPLC method with UV detection and its application to a pharmacokinetic study. Journal of Chromatography B, 859 (1), 69-73). Measurements were repeated 5 times to obtain the most accurate linearity curve on which to base the release studies and wavelength to measure the absorbance. MLX obeyed the Beer-Lambert law in the concentration range of 1-100 μg/mL. MLX solubility in water is 154 μg/mL, so the concentration range was tested upon fell well within this range (71125-38-7 (meloxicam) product description. ChemicalBook. (n.d.). Retrieved from https://www.chemicalbook.com/ChemicalProductProperty_US_CB2191355.aspx).

The UV analysis reached maximum absorbance at 363 nm consistently across all 5 trials, and so was subsequently used for the following release studies.

vii. Release Studies

Triplicates for each pressing method and each loading were included in the release studies to account for variation in release data for each variable. Each MLX loaded sample was placed in 20 mL of phosphate buffered saline (PBS) to mimic sink conditions of a physiological system for the release of the MLX. The samples in the PBS were stored in 20 mL scintillation vials and kept in an incubator at 37° C. until taken out for retrieval of solution at each time point. The timepoints that were used for the release studies ranged from 1-90 days: 1 day, 2 days, 4 days, 7 days, 14 days, 21 days, 28 days, 35 days, 42 days, 49 days, 56 days, and 90 days. When timepoints were taken for each sample, the solution was extracted from the original scintillation vial that had been kept in the incubator and set aside for analysis, keeping the tablet in the original scintillation vial. The scintillation vials were then filled with 20 mL of newly synthesized PBS and put back into the incubator until the next time point.

Once release solutions were retrieved at each time point for the release study samples, the solutions were loaded into a 96 well quartz plate and analyzed using a Spectra Max 384 Plus Ultraviolet Spectrophotometer with SoftMax PRO 4.3.1 software. Samples that did not fall along the previously produced calibration curve were further diluted and reanalyzed using UV analysis. Data was converted into values that considered the various surface areas that were previously measured of the samples due to differences in processing methods. These values were used in analysis of the data.

### Results

i. Morphological Studies

The two-dimensional frames (2-D) from XRD analysis showing the Azimuthal scattering patterns of morphological samples were analyzed visually. The 2-D XRD frames are representative of the morphological makeup of the samples analyzed. These frames are gathered by an x-ray beam being deflected off the sample and into a detector, giving forth to representative bands in each frame (data not shown). The curved bands of light observed, represented the measured peaks of morphological content found within the sample. There were two significant peaks centrally found across all the 2-D frames gathered for this study. These two peaks are representative of the PCL in each sample and are most evident due to the polymer making up the majority of each sample. In the blank PCL sample formulated using the film casting method, the two PCL peaks were sharp and there was some shadowing within the frame. The two sharp peaks indicate the crystalline content of the PCL, while the shadowing indicates the amorphous content, indicative of the natural semi-crystalline nature of PCL. When the blank PCL samples are analyzed after the subsequent processing methods, there is a broadening of the peaks, indicating a change in the crystalline content of the samples and therefore morphological change.

When looking at the MLX-loaded film casted samples, there is evidence of crystallinity as well, with sharp peaks not only for the PCL but evidence of sharp peaks of the MLX also. These MLX peaks are found to the sides of the two central PCL peaks. As the drug loading increases amongst the film casted samples, the MLX peaks become more pronounced and numerous, indicating the MLX has some effect on the morphology of the samples. As with the blank PCL samples, the MLX-loaded samples produce broader peaks once they have undergone the different processing methods. This again indicates a change in morphology for the sample.

To more accurately look at a comparison of the different processing methods across the samples, the 2-D frames were translated into a graphical format using Da Vinci software (FIGS. 3A-3D).

The XRD results now in graphical format reflect the same differences found in the 2-D frames explained previously. The two central PCL peaks are found across all samples and can be seen to broaden with varying degree as different processing methods are applied to the samples. In addition to this, the MLX peaks are found again to the sides of the central PCL peaks and increase in number and magnitude as the MLX loading increases. Again, the broadening of the PCL peaks in addition to the inclusion of the MLX peaks in the samples indicate a morphological change has occurred due to each processing method. To quantify the mesomorphic content found in each sample, deconvolution was performed on each XRD graph in which Gaussian curves were fitted to the PCL peaks.

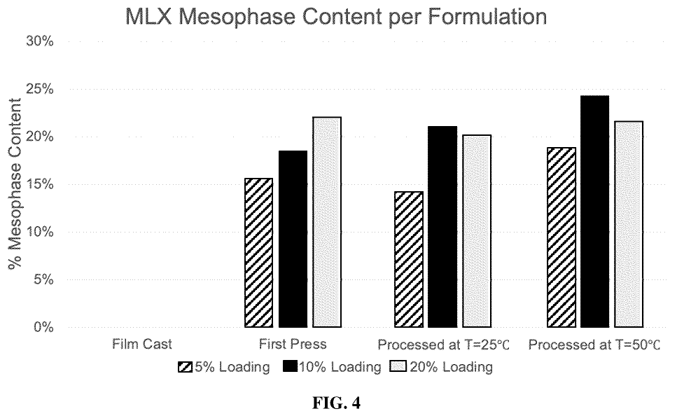

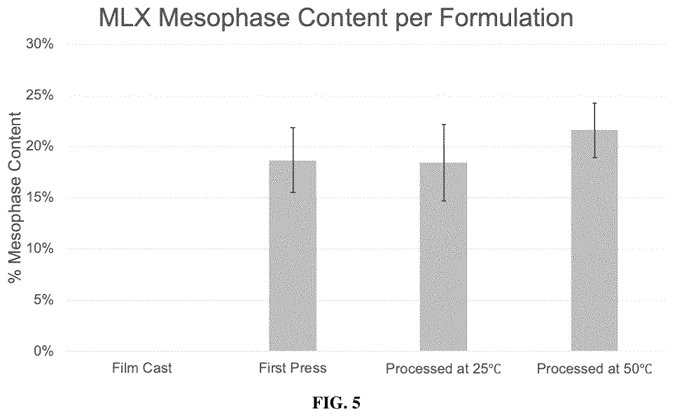

The average mesomorphic content in each sample derived from the deconvolution of the XRD data is shown in FIG. 4. Mesomorphic content was successfully produced in all pressed samples excluding the film cast control where no pressure was applied. The mesophase content of the 5% w/w loaded samples were lower than that of the other loaded samples for each processing method. Using the first press method, each increase in drug loading provided a higher mesophase content percentage on average. For samples that were processed at 25° C. and 50° C., the 10% w/w loaded samples were higher in mesomorphic content on average, while the 5% w/w loaded samples were lowest. However, when all day 0 MLX loaded samples were averaged and compared by processing methods, there were no significant differences found between the mesomorphic content of the various pressed samples (FIG. 5). All samples showed an average of at least 13% induced mesomorphic content across all processing methods.

Once confirmed that the samples contained mesomorphic content after processing, they were studied for mesophase stability over time. Because the different processing methods showed no significant difference in induced mesomorphic content, only 10% w/w MLX loaded samples processed at 25° C. and 50° C. were used in this part of the study. Samples of blank PCL also processed at 25° C. and 50° C. were studied as well to provide a comparison to the MLX-loaded samples. The samples were analyzed using XRD and deconvolution throughout a 90-day period. When using XRD, three different scans in different locations on each sample were gathered and averaged to consider the variation in morphology across each sample.

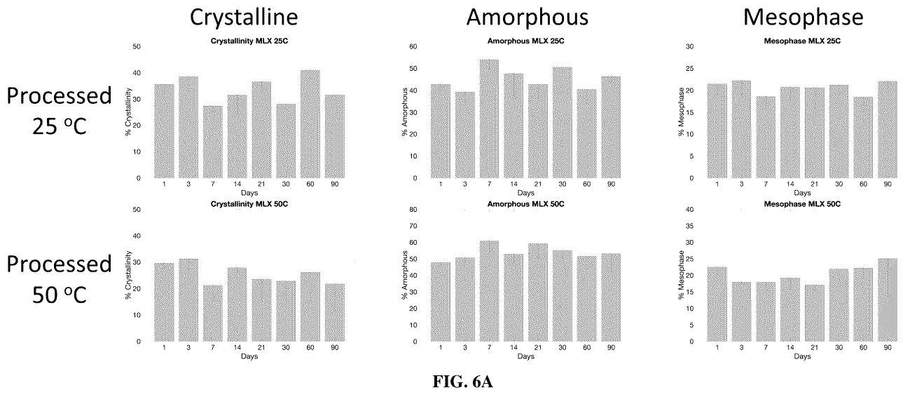

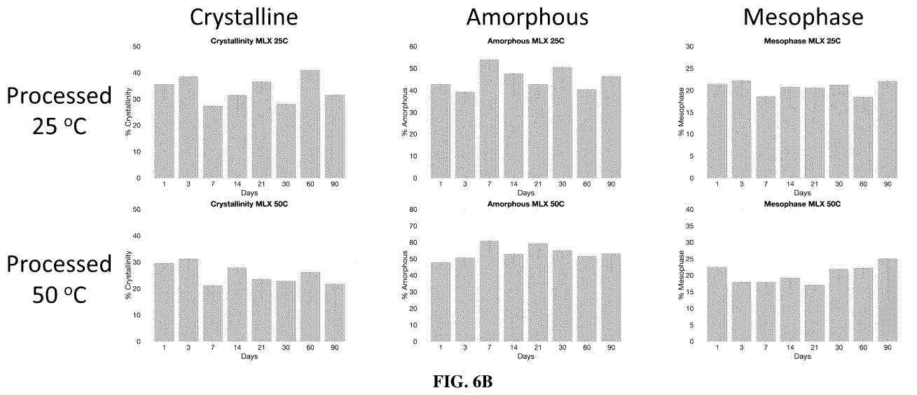

Mesophase content in the samples were consistent with the previous mesophase content amongst the day 0 samples (FIG. 5, FIG. 6A, and FIG. 6B). Mesophase content stayed constant across the entire 90-day period for MLX loaded samples processed at 25° C. and 50° C. The amorphous and crystalline phases of the samples were more variable than the mesophase content over time.

The average mesophase content of the MLX loaded samples (FIG. 6A) showed no significant difference to the mesophase content of the blank PCL samples over time (FIG. 6A), where the blank PCL showed less variability in mesophase content (FIG. 6A).

ii. Release Studies

The MLX loaded samples were processed through the first press, 25° C., and 50° C., procedures in triplicates and contained to release in 20 mL of PBS over a period of 1-90 days. Using UV analysis of the extracted release solution, the cumulative release of drug over time was plotted for the effect of each processing method on the different MLX loadings.

Across all samples displayed in the cumulative release graphs, there is a distinctively sharp increase in MLX released from days 0-7. After this initial spike in release, there is a slow increase in MLX released across the samples, and from the 60- to 90-day period the drug release appears to be leveling off. These characteristics of the release data are indicative of a long-term drug release mechanism. Amongst the 5% w/w MLX-loaded samples, there are no significant differences between the processing methods and their effect on release. However, the drug released across all 5% w/w MLX-loaded samples has not even reached 50% of drug released, indicating a slow-release of drug over time, and implying that the samples could continue to release MLX past the period of 90 days. The 10% w/w and 20% w/w MLX-loaded samples released even slower than the 5% w/w MLX-loaded samples, with none of the 10% w/w or 20% w/w loaded samples reaching even 35% over the 90-day period. The 10% w/w loaded samples showed that the first press method released slower than both second press processing methods, with there being no difference between the second press methods on release. The 20% w/w loaded samples showed this same pattern. Overall, the 10% w/w loaded samples released the slowest amongst the different loadings, and the 5% w/w samples released the fastest.

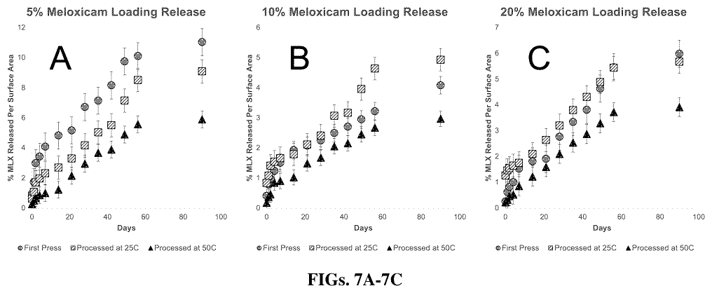

To account of the variance in shape of the samples due to the different processing methods, the release data was normalized by dividing the cumulative MLX release data by each sample's surface area. This release data was again plotted for the effect of each processing method on the different MLX loadings (FIGS. 7A-7C). At the end of 100 days less than 10% of the drug was released which indicates the potential for using such formulation for prolonged time, 6 months and even longer.

According to the release data when normalized for surface area, all MLX loaded samples showed the 50-Cprocessing method to have the slowest release of drug across the 90 days on average. In the 5% w/w loaded sample normalized data, the first press method released quicker than the other two processing methods on average. The 10% w/w loaded normalized release was different in that the 25-° C. processing method released faster than the first press and 50° C. processing methods. The 20% w/w loaded normalized release had similar release profiles for both first press and 25-° C. processing methods with no significant difference. Again, the 10% w/w loaded samples released the slowest across the different drug loadings, with the 5% w/w loaded release being the fastest. This is a very interesting finding and shows that samples with higher drug loading can be fabricated, and still display a slower release rate.

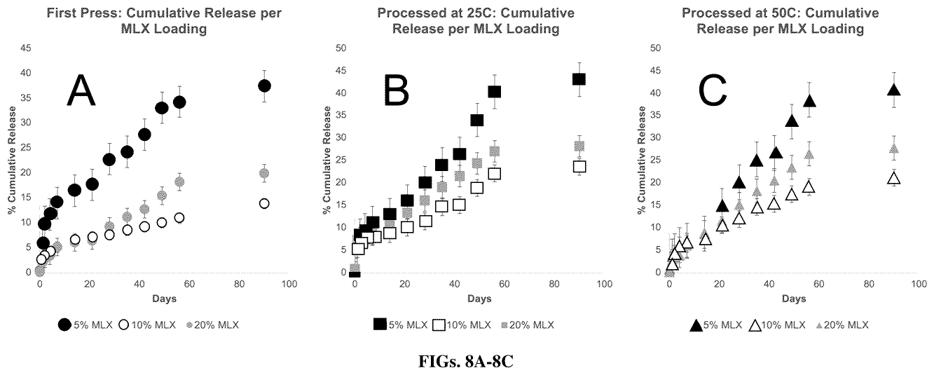

An additional display of release data was created to account for the differences in surface area across the varied processing methods, however, comparing each processing method for the three drug-loaded formulations (FIGS. 8A-8C). Amongst the first press samples, the 5% w/w loaded samples released significantly faster than the 10% and 20% loaded samples. The 10% w/w loaded samples released the slowest within the first press samples. Additionally, this pattern carries on throughout the other two processing methods. Overall, the first press samples released the slowest amongst the different processing methods.

The first aim of the studies in Example 1 was to induce mesomorphic content in a formulation containing both PCL and a drug, MLX, to build upon previous studies in the Mathiowitz Laboratory in which mesomorphic content was induced in PCL (Baptista, C., Azagury, A., Shin, H., Baker, C. M., Ly, E., Lee, R., & Mathiowitz, E. (2020). The effect of temperature and pressure on polycaprolactone morphology. Polymer, 191, 122227. https://doi.org/10.1016/j.polymer.2020.122227). To carry out this part of the project, morphological studies were conducted to demonstrate that mesomorphic content had been induced in all three different processing methods used in the study. The mesomorphic content found in the samples did not differ significantly when compared across processing methods, however there was a trend of increased mesomorphic content in the samples processed at 50° C. that could be expanded upon in future studies. The processed samples indicated over a-15% increase in mesomorphic content on average when compared to the control of film casted samples in which no mesophase occurs. Additionally, the processed samples were tested for how the mesomorphic content was maintained over time through a 90-day period. Mesomorphic content in the samples were constant over the entire 90-day period, giving evidence to the stability of the mesophase over 90 days. While the mesophase content was stable, the amorphous and crystalline content of the samples were more variable. The stability of the mesophase is important for the development of drug delivery systems.

The second aim of the studies in this Example focused on providing evidence that the processed samples could slowly release MLX over time. The release studies conducted during the project used numerous samples spread across three different drug loadings, 5% w/w, 10% w/w, and 20% w/w, and three different processing methods, first press, processed at 25° C., and processed at 50° C. The release of MLX was followed over a 90-day period, in which none of the samples fully released all the drug. In fact, none of the samples reached even 50% of MLX released. This means that the samples could undergo further release for a longer period, such as a year, indicating that a long-term drug delivery vehicle had been formulated. The release data was further analyzed, comparing the effects of the different drug loadings and processing methods on the release kinetics. The first press processing method had the slowest release across all drug-loaded samples when compared to the other two processing methods. This may be evidence that the pressure applied to the samples was the main contributing factor in creating a slow-releasing formulation.

### Example 2. The Impact of Processing-Induced Polycaprolactone Mesophases on the Controlled Release of Dexamethasone

### Materials and Methods

i. Preparation of Solvent Casted DEX and PCL Films

CAPA 640 PCL (Perstorp Holding AB, Molecular Weight: 37 kg/mol), dexamethasone (Sigma, 97% purity), and Dichloromethane (CH₂Cl₂, Sigma) were the starting materials. Primarily PCL films with 5% w/w, 10% w/w, and 20% w/w loading of dexamethasone (DEX) and 250 mg total mass were prepared using the solvent casting method. Appropriate amount of the drug and polymer were weighed and dissolved in 2 mL of Dichloromethane (DCM). To encourage homogenization with high degree of precision, sonication and vortexing was carried out for a total of 15 minutes. The homogeneous mixture was pipetted into a 62 mm petri dish and the solvent was evaporated for 24 hours. The resulting thin film containing the drug and the polymer was peeled from the petri dish and stored at room temperature for further processing. Blank PCL films without the drug, used as the control to compare morphological changes, were prepared using a similar method.

ii. Processing of Solvent Casted Films

Solvent cast films were pressed at 10,000 lbs pressure for five minutes using a manual laboratory press (Specac Inc) and a 20 mm press die at room temperature. The cylindrical tablets for each loading condition were further processed using a Carver Auto Series Hydraulic Press (Carver, Wabash, Indiana) with a heating/cooling system. Samples were placed between two stainless steel 4″×4″ plates and heated at 25 or 50° C. for a total of 10 minutes. The final processing steps involved the application of 20,000 lbs pressure for 5 minutes, followed by rapid cooling of the hydraulic press at the specified pressure. Blank PCL samples were processed using a similar methodology. The surface and drug area density were accounted for by measuring the radius, thickness, and final weight of the tablets. The initial drug loading of the tablets was assumed to be constant post-processing.

iii. Calibration Curves for DEX and PCL

DEX with concentrations ranging from 250 μg/ml to 1 μg/ml was dissolved in phosphate buffer saline (PBS) with a pH of 7.4 using serial dilutions (saturation solubility of DEX in PBS=0.1 mg/mL). Drug absorbance across the solutions from 258-262 nm was measured five times using spectroscopic analysis via Spectra max 384 Plus Ultraviolet Spectrophotometer. Maximum absorbance for DEX occurred at 260 nm and provided the most consistent slope and r-squared for the linearity curve. DEX followed the Beer-Lambert law, and the measurements were in the concentration range of 1-50 μg/mL.

iv. Drug Release

Sink conditions in the physiological environment were mimicked via incubation of the tablets at 37° C. in 20 mL scintillation vials containing PBS. Samples from the PBS solutions were collected at eight different time points (24 Hours, 72 Hours, 1 Week, 2 Weeks, 3 Weeks, 1 Month, 2 Months, 3 Months) and analyzed via UV Spectroscopy. After each time point and solution replacement with fresh PBS, the samples were re-incubated. Drug and monomer concentrations were quantified using the absorbance values obtained from the SoftMax PRO software 4.3.1 from the Spectra max 384 Plus Ultraviolet Spectrophotometer. Concentrations at different time points were calculated using the absorbance values of the calibration curves. Samples with calculated concentrations outside the linearity region were diluted and reanalyzed on the Spectrophotometer.

v. Morphological Studies
a. Preparation of 10% w/w DEX Tablets and Blank PCL Tablets

PCT with 10% w/w DEX loading and blank PCL tablets were prepared using the methodology described for simple release. Processed tablets were then placed into plastic falcon tubes with 20 mL of PBS and incubated at 37° C. Tablets from the eight different timepoints (24 Hours, 72 Hours, 1 Week, 2 Weeks, 3 Weeks, 1 Month, 2 Months, 3 Months) were extracted, lyophilized, and stored at 4° C. until X-ray diffraction (XRD) analysis. About 60 samples were produced for this study. Samples were prepared under various conditions and exposed to aqueous conditions for 90 days and the morphologies of the samples were evaluated by X-ray, to ascertain the stability of the mesophase at 37° C.

b. XRD Analysis

Lyophilized samples were analyzed using a Bruker D-8 Advance X-Ray Powder Diffraction system operating on Da Vinci Software, a Cu X-Ray tube at 40 kV and 40 mA (1600 W), and a Bruker Vantec-500 Xe—CO2 gas-filled detector. The detector had a 13.5 cm diameter window set at 20 cm from the goniometer center. The selected system wavelength was 0.15418 nm. The poly cap was adjusted to a beam divergence of 0.25° for Cu, a spot diameter of less than 4.0 mm, and a 79 mm long 0.3 mm collimator on the incident beam path. All scans ran at a 2θ angle from 20° to 80°, with a step size of 15° and a counting timestep of 60 seconds (300 s total). XRD scans from three different spots in each tablet were obtained to display a more accurate picture of the polymer morphology and dispersion of the drug in the delivery system.

c. XRD Phase Content Calculation

The phase content of each sample was calculated from the obtained XRD scattering profiles by peak deconvolution using the Fityk v1.3.1 software. The deconvolution adapted from Stoclet et al. assumed Gaussian profiles for the scattered peaks and halos. Five peaks were used to characterize the PCL peaks converging approximately at 20 values of 21.4° and 23.8°. Smaller PCL peaks, which can vary in intensity depending on the apparatus resolution, were omitted in the analysis. Gaussian peak 1 characterized the amorphous regions. Gaussian peaks 2 and 3 represented the (110) and (200) orthorhombic crystalline regions. Gaussian peaks 4 and 5, representing the area under the mesophase regions, were added to fit our computed model with the experimental data (R² value: 0.97-0.99). The content of each phase (amorphous, crystalline, and mesophase) was calculated by the area under the diffraction peaks, using the ratio from phase one to the total scattering peaks.

### Results

i. Morphological Studies

The diffusion and rate of release of small molecule drugs are affected by the structure of the polymeric matrix used. Studies show that changing the morphology of the polymer through standard polymer processing techniques such as increasing crosslinking or branching can change the diffusion coefficient of the solute (Liang, Y., Tang, H., Zhong, G. & Li, Z. Formation of Poly(L-lactide) mesophase and its chain mobility dependent kinetics. Chin. J. Polym. Sci. 32, 1176-1187 (2014)). The free volume within the morphological structure affects the change in the diffusion coefficient, enhancing or hindering the overall diffusion rate (Stoclet, G., Seguela, R., Lefebvre, J.-M. & Rochas, C. New Insights on the Strain-Induced Mesophase of Poly(d,1-lactide): In Situ WAXS and DSC Study of the Thermo-Mechanical Stability. Macromolecules 43, 7228-7237 (2010)). The morphology of the polymer at different processing steps was analyzed using XRD analysis to gain an insight into the effect of morphology on the diffusion coefficients.

XRD diffraction profiles of the film cast, first press, and processed tablets were analyzed. According to Wunderlich's definitions of orientational, positional, and confirmational order, the film cast exhibits a diffuse amorphous halo and sharp crystalline peaks characterized by an anisotropic scattering profile (Wunderlich, B. A classification of molecules, phases, and transitions as recognized by thermal analysis. Thermochim. Acta 340-341, 37-52 (1999)). Positional order refers to the intermolecular spacing of the polymeric chains, distinguishable by the tight packing of the crystalline structure and the XRD 20 value of the Debye-Scherrer ring (i.e., isotropic or anisotropic) appearance. The orientational order is associated with the long-range order of the molecular chains, marked by the uniform scattering of the Debye-Scherrer rings. The sharpness of the Debye-Scherrer ring is characteristic of the confirmational order, which further refers to the rotational arrangement of the atoms in a single polymeric chain.

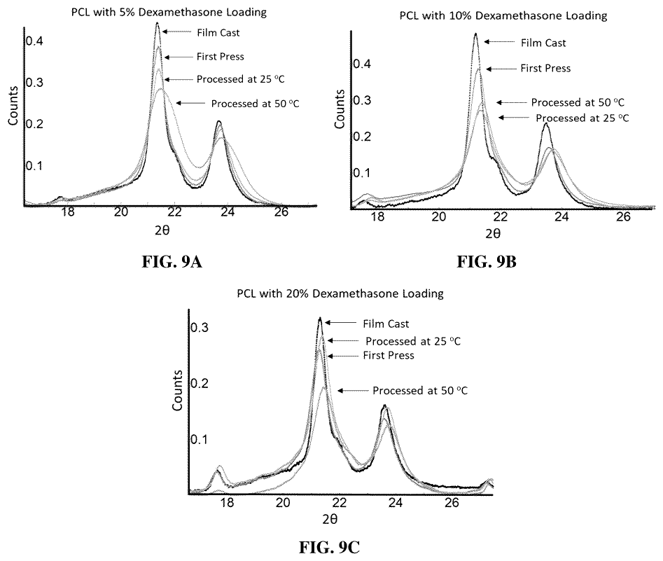

No positional, orientational, or conformational order were observed in the solvent film cast PCL, as demonstrated by weak and diffused scattering of observed crystal diffraction ring. The crystalline phase of the film cast PCL displays the characteristic three levels of positional order associated with the (110), (111), and (200) orthorhombic crystal faces with their inherent conformational order. These three levels of positional order are associated with the intermolecular spacing specific to each crystal face. Based on the XRD profiles and the area normalized integrated scattering patterns in FIGS. 9A-9C, there is a peak broadening of the (110) and (200) Debye-Scherrer rings in the first press and processed samples compared to the film cast. A decrease in crystal size or an increase in structural disorder can be the primary reason for the observed broadening in the crystalline peaks. Since the processing temperature was below PCL's melting temperature (Tₘ=60° C.), the increase in structural disorder and plastic deformation of the initial orthorhombic crystal due to pressure application most likely caused the broadening.

Hence, the morphological characteristics observed in this PCL-based system are comparable to the plastic crystal mesophase. Plastic crystal mesophases form due to crystallite deformation and slip planes within the molecular structure. These crystals exist in a “plastic” state with positional order, exhibiting disorder in both conformational and orientational order. Due to the long-chain hydrocarbon structure of PCL, orthorhombic structural dislocations or several screw and edge dislocations can occur on either side of the methyl groups (Rezgui, F., Swistek, M., Hiver, J. M., G'Sell, C. & Sadoun, T. Deformation and damage upon stretching of degradable polymers (PLA and PCL). Polymer 46, 7370-7385 (2005)). This further causes the slipping and sliding of the individual crystal layers. The irregularly attached carbonyl groups along the chain direction also can contribute to the disordering of the crystal, allowing the formation of the plastic crystal mesophase. Apart from plastic mesophases, a partial or complete conformational disorder with birefringent properties associated with the positional and orientational order characterize condis crystal mesophases. Previous studies show that a temperature range between the onset and offset of the PCL melt (50-60° C.) leads to the formation of a condis crystal mesophase (Baptista, C. et al. The effect of temperature and pressure on polycaprolactone morphology. Polymer 191, 122227 (2020)). Peak deconvolution on the dexamethasone-loaded PCL tablets was conducted to analyze the morphological contents of the system quantitatively. Stoclet et al.'s method of using Gaussian profiles for all scattered peaks and halos was adapted to account for the crystalline, amorphous, or mesophase content (Stoclet, G., Seguela, R., Lefebvre, J.-M. & Rochas, C. New Insights on the Strain-Induced Mesophase of Poly(d,1-lactide): In Situ WAXS and DSC Study of the Thermo-Mechanical Stability. Macromolecules 43, 7228-7237 (2010)). The ratio between the peak area and the total scattering area was used for the calculations. The amorphous halo parameter was set to the recurring 20 value from the unaltered PCL at approximately 21°. The (110), (111), and (200) orthorhombic crystalline peaks were characterized, and curve fitting of the mesophase peaks was conducted using an R² value of at least 0.98.

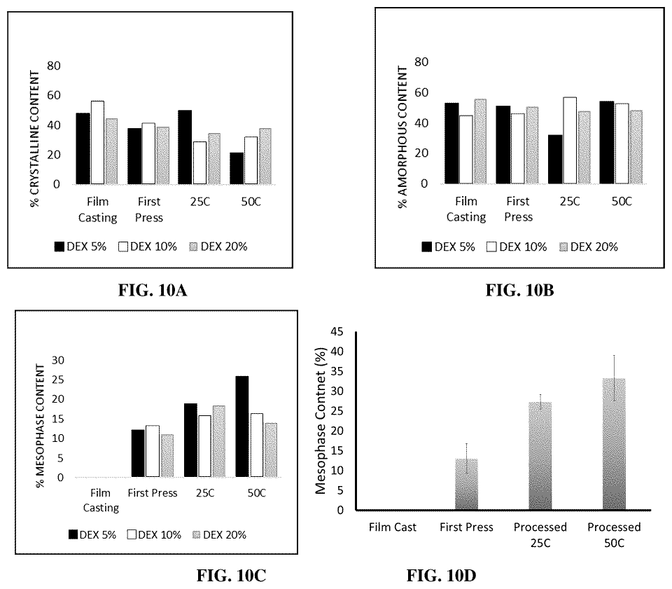

As seen in FIGS. 10A-10C, a general trend can be observed in the mesophase content for PCL tablets loaded with 5% w/w dexamethasone. Increasing processing force, temperature, and plastic deformation correlate to increased percent mesophase. Since an R² value of at least 0.98 was achieved in the film cast samples without adding the mesophase Gaussian peaks, it was assumed that the solvent film cast PCL samples display a typical semi-crystalline morphology with no detectable mesophase. The 13% increase in the mesophase content between the first press and processed 50 C 5% w/w DEX tablets is correlated with a 9% decrease in crystallinity. The previously reported mechanism may explain this finding that the formation of the mesophase and the observed broadening of the peaks result from plastic deformation and crystal disorganization. If the mesophase variation between 5% w/w, 10% w/w, and 20% w/w drug loading is discounted (FIG. 10D), a general trend is observed in blank PCL which increasing the force and temperature is directly correlated to an increase in mesophase formation (FIG. 10D). For the samples made with drugs (FIG. 10C) the trend is not as pronounced.

The film cast samples were processed with no pressure or heat application via a solvent casting method. These samples were further processed to make the ‘first press’ samples by compression via a hand-held press at 10,000 lbf. The initial pressure application in the restricted environment of the compression mold die, causes the formation of the mesophase (calculated on the order of 8-17%). Finally, the processed samples at 25° C. and 50° C. were produced from the ‘first press’ samples, by pressing in a temperature controlled hydraulic press under 20,000 lb_{f}. In these samples, the environment was not restricted to a compression die, but rather upon application of force the samples were subject to plastic deformation and extruded flow between the plates of the hydraulic press. As a result, increased mesophase for DEX 5% w/w (calculated on the order of 25-40%) was estimated from the XRD deconvolution. For tablets with higher drug loading, in the second press, despite the extruded flow and increased pressure application, the mesophase produced remained constant after the first press. It is believed that the crystallinity of the drug is interfering with the formation of the mesophase despite additional processing.

Tablets taken out at specific time points (in the morphology study) were analyzed to assess the stability of the system over time in the release medium. Since it was previously assumed that the induction of extruded flow, increased temperature and pressure application will result in the maximum mesophase formation, only DEX tablets with 10% w/w loading processed at 25° C. and 50° C. were analyzed. Blank PCL tablets without the drug were processed similarly and used as controls to determine if the stability of the system changes with the presence of the drug over time. Three spots per tablet were evaluated to account for the uneven dispersion of the drug in the polymeric matrix. As seen by the dispersion of peaks calculated via Gaussian curve fitting in, no significant differences were observed in the morphological content for blank PCL or drug loaded tablets over a span of 3 months. This further indicates that the birefringently formed mesophase is stable at 37° C. in the release medium, increasing the system's applicability for long term-controlled release devices.

ii. In Vitro Release

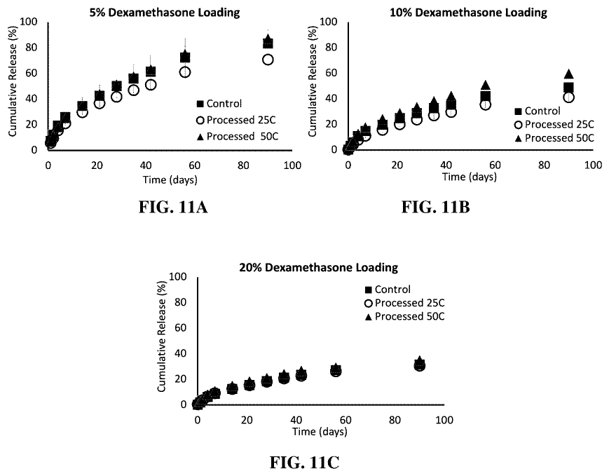

The effect of morphology and processing conditions on the release kinetics was analyzed by suspending tablets loaded with Dexamethasone in PBS at 37° C. Sustained release over a 90-day duration was achieved for all tablets, independent of processing conditions and percent drug loading (FIGS. 11A-11C). An increase in drug loading from 5% w/w, 10% w/w, to 20% w/w was correlated with a decrease in cumulative drug release. This trend may be explained by the low saturation solubility of Dexamethasone in the release medium (0.1 mg/mL) (Colombo, M., Staufenbiel, S., Rühl, E. & Bodmeier, R. In situ determination of the saturation solubility of nanocrystals of poorly soluble drugs for dermal application. Int. J. Pharm. 521, 156-166 (2017)). Due to the high concentration of Dexamethasone in all processed tablets, the saturation solubility might act as the rate limiting step. Typically, in systems where the drug concentration is lower than the saturation solubility, an increase in drug loading would correlate with an increase in percent drug released over time (Savjani, K. T., Gajjar, A. K. & Savjani, J. K. Drug Solubility: Importance and Enhancement Techniques. ISRN Pharm. 2012, 195727 (2012)).

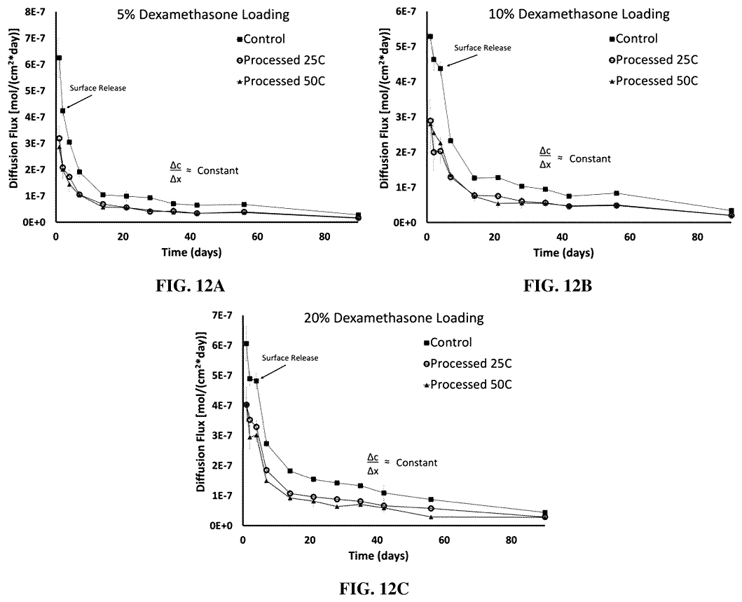

As mentioned earlier, due to its hydrophobicity PCL has a degradation profile lasting up to 2-4 years (Polycaprolactone—an overview|ScienceDirect Topics. https://www.sciencedirect.com/topics/chemical-engineering/polycaprolactone). After 3 months of release, the mass variation of the formulation was less than 2% and no significant signs of erosion were observed. This confirms that the release of dexamethasone through the polymeric matrix occurred primarily through diffusion, following Fick's law. FIGS. 12A-12C portray the change in calculated diffusion flux over time. For all three scenarios the diffusion flux through the polymeric matrix is high in the first week. It continues to decline for a period of two weeks before reaching a constant flux. This phenomenon can be explained by the Higuchi pseudo steady state model (Reynolds, T. D., Mitchell, S. A. & Balwinski, K. M. Investigation of the effect of tablet surface area/volume on drug release from hydroxypropylmethylcellulose controlled-release matrix tablets. Drug Dev. Ind. Pharm. 28, 457-466 (2002)). The release model used to describe drug release from thin films in sink conditions makes the following assumptions in this scenario:
1) There is rapid diffusion through the surface, but the rate limiting step is the diffusion of the drug through the PCL matrix.
2) The concentration of Dexamethasone in the release medium is assumed to be negligible and the system behaves like a perfect sink.
3) Dexamethasone is finely dispersed and homogenously distributed through the polymeric matrix.
4) The concentration of Dexamethasone in the PCL matrix is higher than the saturation solubility of the drug in PBS on day 0.
5) There is minimal swelling and dissolution of PCL during drug release.
6) The diffusion of the drug is limited to a single dimension and the release from the edge is negligible.
7) The diffusion coefficient of Dexamethasone release through PCL is constant and is not time or position dependent.
8) Drug dissolution through the matrix is faster than drug diffusion.

Given these conditions, the release kinetics observed in FIGS. 12A-12C can be explained by the schematic of the Higuchi model. The initial release from the PCL occurs close to the surface of the tablet through diffusion in sink conditions. As a result, during the first week of release, when the drug's surface concentration is at saturation, a rapid diffusion flux is detected. It is believed that drug diffusion through the PCL matrix has no impact on the release rate at these time points and that surface dissolution is the only regulating factor. After the rapid dissolution through the surface, the concentration of the dissolved drug molecules at the surface starts falling below the 0.1 mg/mL saturation concentration of dexamethasone. Based on an inward shifting concentration gradient, the dissolved drug molecules positioned further away from the PCL surface then begin to permeate through the tablet matrix into the release medium. The Higuchi model assumes pseudo steady state behavior in which rapid dissolution causes the dissolved drug concentration gradient to be maintained at saturation concentration within the PCL matrix. The concentration gradient at the surface is assumed to be zero due to rapid release. The assumption can be confirmed by the kinetics observed in FIGS. 12A-12C, where the moving diffusion front and thickness of PCL have a negligible effect on the concentration gradient after the initial rapid surface release.

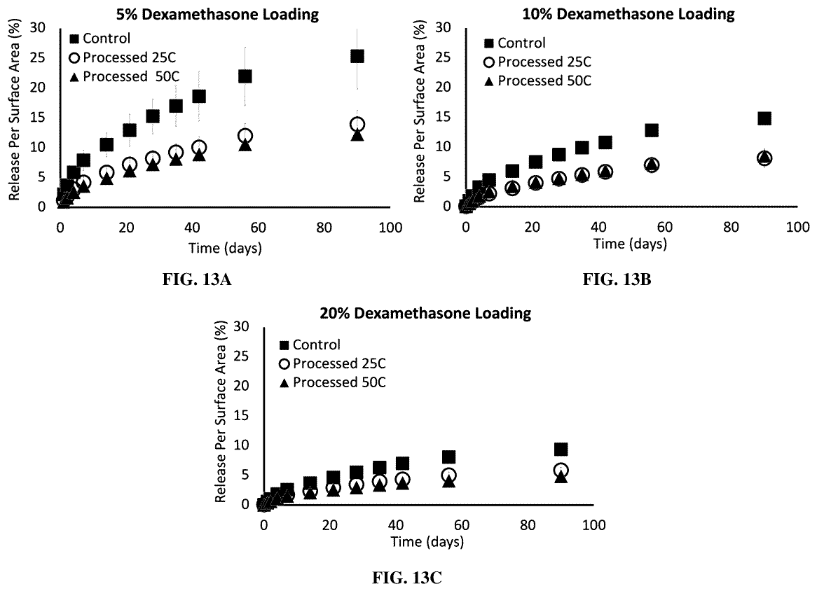

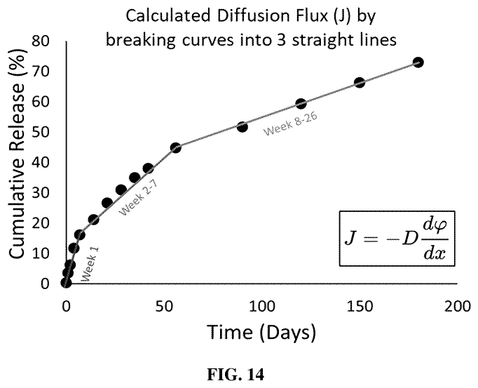

The analysis could be further improved by considering the variability in the surface area of the tablets associated with differences in the processing conditions at each step. Despite absence of significant differences in the total cumulative release between the first press and processed tablets, when normalized to surface area (FIG. 13), significant differences were detected in the release between first press tablets and tablets processed at 25° C. and 50° C. Usually, an increase in surface area to volume ratio is correlated with an increase in percent release (Reynolds, T. D., Mitchell, S. A. & Balwinski, K. M. Investigation of the effect of tablet surface area/volume on drug release from hydroxypropylmethylcellulose controlled-release matrix tablets. Drug Dev. Ind. Pharm. 28, 457-466 (2002)). However, change in surface area does not seem to have an impact on the cumulative release (FIG. 11). A decrease in percent release per surface area is detected with increasing drug loading (FIG. 13). To analyze the differences observed further, the diffusion flux assumed to be constant in week 1, weeks 2-7 and weeks 8-26 (FIG. 14) was calculated. Fick's first law of diffusion was used to calculate the values using the following equation (33):

$$J = - D \frac{Δ C}{Δ x} \qquad ( 1 )$$

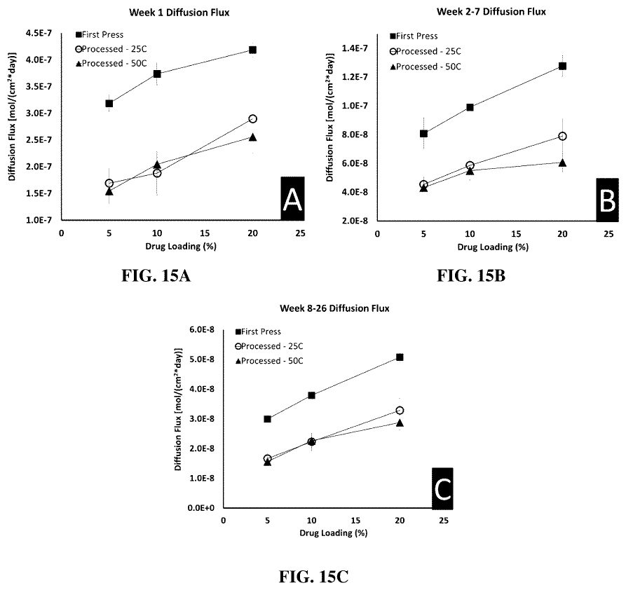

J=Diffusion flux, D=Dexamethasone diffusion coefficient, ΔC/Δx=the concentration gradient of dexamethasone between the PCL surface and diffusion front. For all three drug loadings, the calculated diffusion flux for the samples processed at 25° C. and 50° C. was lower than the diffusion flux for the first press samples (FIG. 15). The combined observations in FIG. 15 and FIGS. 12A-12D indicate that the differences in flux recorded are an outcome of the variable diffusion coefficients of the drug through the polymeric matrix. The variability in the diffusion coefficients was modeled using the Higuchi equation (Brophy, M. R. & Deasy, P. B. Application of the Higuchi model for drug release from dispersed matrices to particles of general shape. Int. J. Pharm. 37, 41-47 (1987)):

$$\frac{{M}_{t}}{A} = \sqrt{2 {Dc}_{ini} {c}_{s} t} \qquad ( 2 )$$

$$Solving for D :$$

$$D = {( \frac{{M}_{t}}{A \sqrt{2 {c}_{ini} {c}_{s} t}} )}^{2} \qquad ( 3 )$$

$$Solving for {M}_{t} :$$

$${M}_{t} = A \sqrt{2 {Dc}_{ini} {c}_{s} t} \qquad ( 4 )$$

Mₜ=cumulative mass of drug release at time t, A=total surface area of the tablet, D=diffusion coefficient of Dexamethasone, cᵢₙᵢ=initial concentration of the drug in PCL, and cₛ=saturation solubility limit of the Dexamethasone in PBS.

The Higuchi modeled release (Mₜ) versus the experimental release data (n=3) was analyzed. With slight deviations towards the end of the release profile for 5% w/w and 10% w/w drug loading, the experimental data matches the modeled release with a relatively tight fit (R²≈0.93). The deviations can be attributed to the assumption that the model is in a pseudo steady-state with the tablet thickness having a negligible effect on the inward diffusion front. An increase in Δx (distance between the surface of the tablet and the diffusion front of the Higuchi model) would contribute to a decrease in the mass release, assuming a constant concentration gradient. Despite the minimal deviations, the Higuchi model explains the release kinetics observed in the system.

The effect of changing the surface area on the mass release was modeled by keeping the diffusion coefficient constant and modeling the release using the Higuchi equation. Based on the modeled curves for 5% w/w drug loading, an increase in the surface area to volume ratio should yield a higher cumulative release. However, despite the change in the surface area to volume ratio, there was minimal impact on the cumulative mass release. A similar trend was observed for 10% w/w and 20% w/w drug loading.

This investigation illustrates that to appropriately describe the release with the Higuchi equation, a new diffusion coefficient must be produced for each sample.

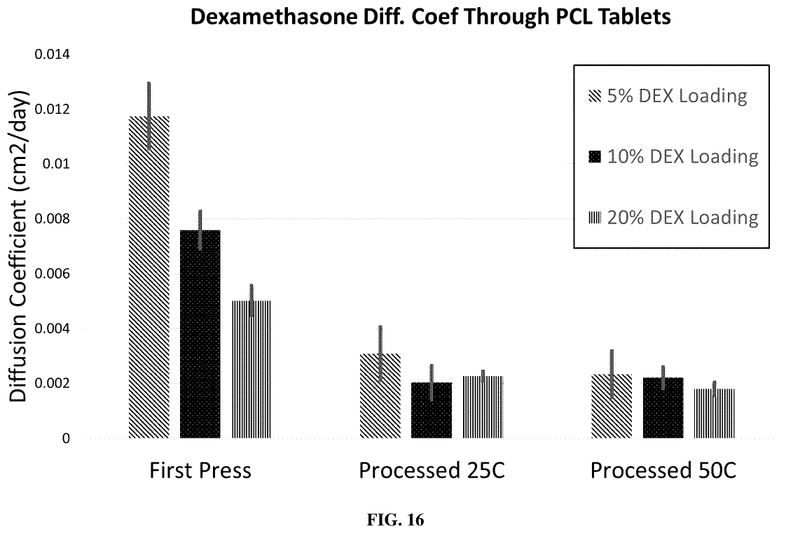

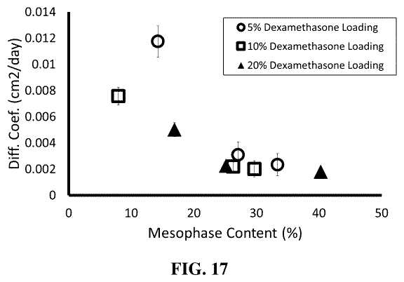

Lastly, the computed diffusion coefficients derived from Equation 3 are illustrated in FIG. 16. Significant differences are observed between the first press and 25° C. and 50° C. processed samples. The processed samples display a lower diffusion coefficient for all three drug loadings. For the first press samples, an increase in the percent loading is correlated with a decrease in the diffusion coefficient. This trend can be explained by the morphological differences observed in the mesophase earlier. As depicted in FIG. 17, samples with a lower mesophase content (first press) have a higher diffusion coefficient and thus display a faster release, whereas samples with a higher mesophase content (processed 25° C. and 50° C.) display a lower diffusion coefficient and slower release.

Not wishing to be bound by theory, it is believed that the release of dexamethasone occurs through the diffusion of the drug through the polymeric matrix. Hence an increase in the density or a decrease in the available free volume of the amorphous region results in a decrease in the diffusion coefficient of the drug. It was previously shown that increasing processing pressure and inducing plastic deformation causes mesophase formation at the expense of both, amorphous and crystalline phases. Hence, the correlation observed in FIG. 17 can be attributed to the decrease in the free volume of the amorphous phase due to plastic or condis crystal mesophase formation. The resultant decrease in the area available for drug diffusion is most likely responsible for the decrease in the diffusion coefficient.

A long-term sustained release system for DEX loaded PCL tablets with 5% w/w, 10% w/w, and 20% w/w drug loading was achieved in this example. Through XRD analysis and peak deconvolution methods, it was shown that the samples processed at a higher temperature and pressure contained more mesophase content (25-40%) than the first press samples (8-17%). The morphological studies further revealed that the system was stable for up to 3 months in the release medium without observable degradation. The release of dexamethasone through the polymeric matrix occurred through Fickian diffusion in a pseudo steady-state manner and was modeled through the Higuchi equation. A variable diffusion coefficient was correlated with a decreased diffusion flux and reduced percent release per surface area of sample. The first press samples processed under lower pressures of 10,000 lb_{f} display a higher rate of release and higher diffusion coefficients (5E-3 to 12E-3 cm²/day) than samples processed at 20,000 lb_{f} (25 or 50° C.) with unrestricted flow (2E-3 to 3E-3 cm²/day). In conclusion, the lower diffusion coefficients are thought to be correlated with the decrease in the free volume associated with mesophase formation. There is strong evidence suggesting that formation of the mesophase can help achieve a long-term sustained release system with a controllable rate of drug diffusion.

### Example 3. The Impact of Processing-Induced Polycaprolactone Mesophases on the Release Kinetics of Dolutegravir

### Materials and Methods

i. Preparation of the Dolutegravir (DTG) and Blank PCL Films

CAPA 640 PCL was received from Perstorp Holding AB (Perstorp, Sweden) for scientific use with a molecular weight (MW) of 37 kg/mol. Primarily PCL films with 5% w/w, 10% w/w, and 20% w/w loading of dolutegravir (DTG) and a total mass of 250 mg per film were prepared using solvent casting. Blank PCL films were only composed of the polymer and prepared with the same procedure. Those films were made by weighing the appropriate amount of PCL and DTG and dissolving them in 2 mL of dichloromethane (DCM). Complete dissolution required a few rounds of sonication and vortex mixing. After the solution becomes completely clear, the contents were pipetted into a 62 mm petri dish. A day later, the solvent completely evaporated and resulting in a thin film containing the drug and the polymer. The same procedure was followed for blank PCL films. These films are referred to as film casts in the analysis.

ii. Processing of the DTG and Blank PCL Films

In a series of pressing steps, films were first all pressed using a hand press and die. The press occurred at 10,000 lbs and lasted for 5 mins at room temperature. All films became cylindrical tablets. For each loading condition and the blank PCL, some of those cylindrical tablets become the first press tablets for the release studies. These are defined as such as the tablets did not go through further processing. The remaining tablets for each loading condition were then pressed using a Carver Auto Series Hydraulic Press (Carver, Wabash, Indiana) equipped with a heating/cooling system. Usually, three tablets were placed between two 4″×4″ stainless steel plates and then heated at 25° C. with the plates of the hydraulic press for 10 min. After heating time, the samples were pressed at 10,000 lbs for 5 mins and immediately after cooled until the plates of the hydraulic press returned to room temperature while still maintaining the pressure. This ensures that the induced morphology is maintained in the PCL after the pressure is removed. The same procedure was followed for the remaining tablets that were pressed at 50° C. in each loading condition. Blank PCL films also had tablets that were pressed using the hydraulic press at 25° C. and 50° C. under 10,000 lbs. These tablets are referred to as “processed 25° C.” and “processed 50° C.”′ in later sections of this example. Once processing was complete, final weight and physical dimensions for each tablet were recorded. Final amounts of drug and polymer in each tablet were calculated assuming that the initial loading remained the same after losses occurred during the solvent evaporation and pressing steps.

iii. Calibration Curves for DTG

DTG was dissolved in phosphate buffer saline (PBS) with a pH of 7.4. The maximum drug solubility was determined as 0.05 mg/mL (50 μg/mL). Through a series of dilutions, DTG solutions with concentrations from 50 μg/mL to 1 μg/mL were prepared. A Spectra max 384 Plus Ultraviolet Spectrophotometer (UV) was used to measure the absorbance of the DTG solutions. DTG is reported to have maximum absorbance at 260 nm¹⁹. Absorbance of the solutions was measured from 258 nm to 262 nm. With the measurements being repeated 3 times, choosing a range ensures that the wavelength chosen for the release studies provides the most consistent slope and r-squared for the linearity curve. DTG was found to obey the Beer-Lambert law in the concentration range of 1-20 μg/mL. The linearity curve was calculated as:

$$y = 0.0044 x + 0.0176 , {R}^{2} = 0.9891 \qquad ( 1 )$$

Based on the data observed, it is possible that this release study may not have full sink conditions.

iv. Drug and Monomer Concentrations

Each tablet was then placed in a scintillation vial containing 20 mL of PBS to mimic sink conditions in a physiological environment. These samples were also incubated at 37° C. for the entirety of the release studies experiments. At specific timepoints, the PBS solutions for each sample were collected and analyzed via UV spectroscopy to quantify the amount of drug or monomer released. Following the collection of PBS solutions, the release medium for each tablet were subsequently changed with fresh PBS and then incubated again until the next collection of PBS solutions. Drug and monomer concentrations were quantified using the absorbance values obtained from the SoftMax PRO software 4.3.1 of the Spectra max 384 Plus Ultraviolet Spectrophotometer. Using the absorbance values and the calibration curves, concentrations were calculated for the different timepoints of the release studies. Solution concentrations usually fell outside the linearity region for DTG and hence, required dilutions. Following dilutions, new absorbance values were obtained, and concentrations were once again calculated using the dilution factor. As PCL can take up to 2 years to degrade, monomer concentrations were usually below the limit of detection. Release studies experiments lasted up to three months.

v. XRD Phase Content Calculation

The phase content of each sample was calculated from the obtained XRD scattering profiles by peak deconvolution using the Fityk v1.3.1 software. The fitting method used was developed by Stoclet et al. in which Gaussian profiles were assumed for all scattering peaks and halos. As such, the fitting involved the use of 5 peaks to characterize the major PCL peaks happening approximately at 20°-values of 21.4° and 23.8°. Other PCL peaks were not included in the analysis as those can greatly vary in intensity depending on the resolution of the apparatus. The first peak was used to characterize the broad amorphous. Two peaks were then added to represent the (110) and (200) orthorhombic crystalline face peaks. Finally, two mesophase peaks were added to fit the computed model with the experimental data with an R² value of at least 0.95. The content of each phase (amorphous, crystalline and mesophase) was calculated by the area under the diffraction peaks, using the ratio from one phase to the total scattering peaks.

vi. Statistical Analysis

ANOVA tests were performed to assess the changes in phase content occurring in the polymer for the morphological studies experiments. Standard deviation error bars were used to assess the repeatability and reproducibility of the release studies for the tablets that were prepared using the exact same protocol. Multiple student t-tests were used to evaluate significance between the calculated diffusion coefficients for each processing condition in a given percent loading.

### Results

i. Morphological Analysis: Time 0 (Day 0)

The diffusion and rate of release of small molecule drugs are affected by the structure of the polymeric matrix used. Studies show that changing the morphology of the polymer through standard polymer processing techniques such as increasing crosslinking or branching can change the diffusion coefficient of the solute (Peppas, N. A. & Lustig, S. R. The Role of Cross-links, Entanglements, and Relaxations of the Macromolecular Carrier in the Diffusional Release of Biologically Active Materials. Ann. N. Y. Acad. Sci. 446, 26-40 (1985)). The free volume within the morphological structure affects the change in the diffusion of the drug, enhancing or hindering the overall release rate. To this end, a significant amount of time was spent in this study, analyzing the morphology of the PCL carrier. Before starting the release studies, a single XRD scan was taken at the different processing steps (film cast, first press, processed tablets) for the blank PCL and drug loaded PCL samples to gain insight into the morphology of the polymer at each of these processing conditions. The 2D XRD azimuthal scattering patterns of the film cast, first press and processed samples were analyzed. As per Wunderlich's definitions of orientational, positional, and conformational order, the film casts for blank PCL, DTG 5% w/w, DTG 10% w/w, and DTG 20% w/w all show a typical isotropic scattering profile characterized by a diffuse amorphous halo and sharp (110), and (200) crystalline peaks. The amorphous phase of the film cast samples displays no positional, orientational or conformational order, hence presenting a weak and diffuse scattering ring that underlies the crystal diffraction. The crystalline phase of the film cast samples displays the characteristic three levels of positional order associated with the (110), and (200) orthorhombic crystal faces with their inherent conformational order. To clarify this analysis, revisiting the definitions of orientational, positional and conformational order set by Wunderlich would be primordial (FIG. 1).

In this instance, orientational order refers to the long-range order of the polymer chains and can be observed by XRD as the uniformity of scattering of a given Debye-Scherrer ring (i.e., isotropic or anisotropic). Positional order refers to the intermolecular spacing, which is typically characteristic of the packing of a particular crystal structure and can be observed by XRD at the 20 value at which a given Debye-Scherrer ring occurs. As mentioned earlier, there are three levels of positional order associated with the (110), and (200) orthorhombic crystals face of PCL. These three levels of positional order are associated with the intermolecular spacing specific to each crystal face. Finally, conformational order refers to the rotational arrangement of the atoms in a single polymer chain (i.e. gauche or anti conformation) and can be observed by XRD as the sharpness of a given Debye-Scherrer ring.

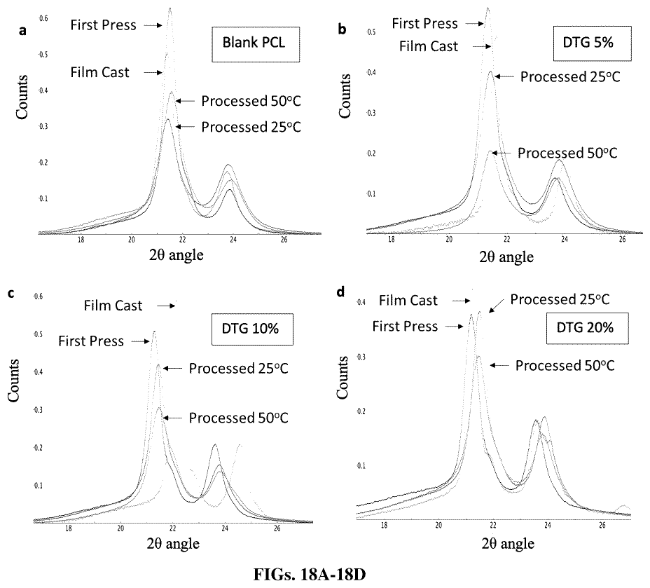

In the first press, processed 25° C. and processed 50° C. tablets, the scattering patterns start to broaden compared to the film cast samples, evidenced by the thickening of the (110) and (200) face Debye-Scherrer rings. This broadening of the (110) and (200) faces can be seen more clearly in the area normalized integrated scattering profiles depicted in FIGS. 18A-18D. Generally, the broadening of a crystalline peak can be primarily attributed to either decreasing crystal size or increasing structural disorder within the sample. Since the processing temperature in all instances was below the Tₘ of PCL, it is most likely that the plastic deformation from applying pressure well below the melting temperature results in a disordering of the initial orthorhombic crystal conformation. Hence, the observed morphology for first press, processed 25° C. and processed 50° C. tablets is that of the plastic crystal mesophase (Baptista, C. et al. The effect of temperature and pressure on polycaprolactone morphology. Polymer 191, 122227 (2020)). Plastic crystals are defined as a type of mesophase in the “plastic” state with positional order and both conformational and orientational disorder (Wunderlich, B. A classification of molecules, phases, and transitions as recognized by thermal analysis. Thermochim. Acta 340-341, 37-52 (1999)). The mechanism of formation of plastic crystals is thought to be the deformation of crystallites, due to a large number of slip planes within the structure (Baptista, C. et al. The effect of temperature and pressure on polycaprolactone morphology. Polymer 191, 122227 (2020)). In the case of PCL, it is known that long chain hydrocarbon crystals of orthorhombic structure can form dislocations on either side of which the terminal methyl groups lie. In addition, several screw and edge dislocations can occur in these long chain hydrocarbon crystals. Any of these dislocations are noted to cause individual layers of the crystal to slip over one another (Keith, H. D. & Passaglia, E. Dislocations in Polymer Crystals. J. Res. Natl. Bur. Stand. Sect. Phys. Chem. 68A, 513-518 (1964)). Alternatively, the disordering of the PCL crystal structure can be attributed to irregularly located carbonyl groups along the chain direction (Hu, H. & Dorset, D. L. Crystal structure of poly(is-caprolactone). Macromolecules 23, 4604-4607 (1990); Chatani, Y., Okita, Y., Tadokoro, H. & Yamashita, Y. Structural Studies of Polyesters. III. Crystal Structure of Poly-8-caprolactone. Polym. J. 1, 555-562 (1970)). These slip-planes coupled with irregularly spaced carbonyl groups are thought to allow for plastic crystal formation in PCL upon plastic deformation (Baptista, C. et al. The effect of temperature and pressure on polycaprolactone morphology. Polymer 191, 122227 (2020)).

For all drug loaded tablets (FIG. 18A-18D), first press, processed 25° C. and processed 50° C. tablets show shorter and broader crystalline peaks as pressure and temperature cause the polymer to lose some of its crystalline content and amorphous content to the mesophase formation (i.e., plastic crystal) of PCL. This effect is also seen in blank PCL even though the first press sample curiously shows a more intense crystalline peak (110) than the film cast. The slight shift of the crystalline peaks observed in FIG. 18C can be due to compression or expansion of the crystal lattice structure. In addition to the plastic crystal, it was also previously reported that at processing temperatures around 50° C. and 60° C. (between the on-set and off-set of the PCL melt), the formation of a condis crystal mesophase could be seen (Baptista, C. et al. The effect of temperature and pressure on polycaprolactone morphology. Polymer 191, 122227 (2020)). Condis crystals are defined as a type of mesophase in the solid state with birefringent properties attributed to positional and orientation order, and partial or full conformational disorder (Wunderlich, B. A classification of molecules, phases, and transitions as recognized by thermal analysis. Thermochim. Acta 340-341, 37-52 (1999); Androsch, R. J., Di Lorenzo, M. & Schick, C. Mesophases in polyethylene, polypropylene, and poly(l-butene). Polymer 51, (2010)). Accordingly, it appeared that when sufficient chain mobility was achieved at increased temperatures of 50° C. and 60° C., condis crystal mesophase is created based on a similar mechanism as the formation of the plastic crystal. While PCL formed a condis crystal mesophase around such processing temperatures, the presence of DTG seems to prevent the formation of such a phase. Indeed, condis crystal was characterized by two broadened and anisotropic arcs around the equator in azimuthal scattering patterns. The focus on the equator is owed to the orientation in the radial direction (perpendicular to the direction of the sample compression). None of the samples processed at 50° C. showed formation of the condis crystal phase, but still displayed properties of the plastic crystal mesophase. Overall, the first press, processed 25° C., and processed 50° C. tablets showed the presence of the plastic crystal mesophase. With this qualitative analysis, a quantitative approach is required to distinguish the morphological differences resulting from the different processing conditions, discussed in the ensuing paragraphs.

A quantitative structural analysis of the blank PCL and DTG-loaded PCL samples were obtained by peak deconvolution of the integrated XRD scattering profiles.

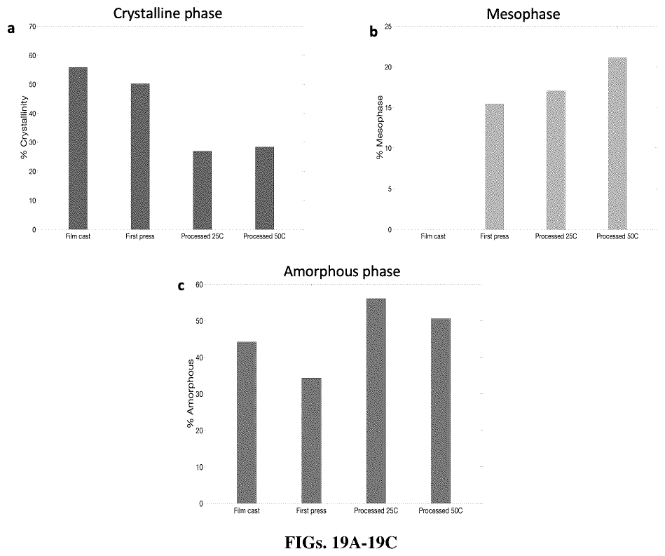

Specifically, the crystalline, amorphous, and mesophase phase content of each sample was calculated based on the peak area contribution to the total scattering area, using the methods described by Stoclet et. al. (Stoclet, G., Seguela, R., Lefebvre, J. M., Elkoun, S. & Vanmansart, C. Strain-Induced Molecular Ordering in Polylactide upon Uniaxial Stretching. Macromolecules 43, 1488-1498 (2010)). Additional details of this method applied to PCL have been previously reported (17). The deconvolution approach assumed Gaussian profiles for all scattering peaks and halos. Particularly, the amorphous halo parameters were determined by the unaltered PCL which displayed the halo peak at a 20 value of approximately 21°. Upon establishing a best fit amorphous halo, the (110), and (200) orthorhombic crystalline face peaks were established. Finally, mesophase peaks were fit to a R² value of at least 0.95 when comparing the computed model to the experimental data. The content of each phase (amorphous, mesophase, and crystalline) was calculated by the area under the diffraction peaks, using the ratio from one phase to the total scattering peaks. The calculated deconvolution values for blank PCL for each manufacturing step are shown in FIGS. 19A-19C. FIG. 19A shows a decrease in percent crystallinity in going from film cast to the first press and finally to the processed 25° C. samples. Processed 50° C. samples seem to have about the same percent crystallinity as processed 25° C. samples. Mesophase content shows an increasing trend from the first press samples to the processed 25° C. and finally to the processed 50° C. samples. Film cast samples did not show the presence of mesophase as discussed above (FIGS. 18A-18D) and hence have no detectable mesophase content (0%). Finally, the amorphous phase content does not seem to show any trend from the beginning manufacturing step (film cast) to the first press or processed samples. However, it is important to note that processed samples have higher percent amorphous than first press or film cast samples.

Like the blank PCL samples, the crystalline, amorphous and mesophase contents for DTG loaded PCL tablets were also analyzed. For all three loadings, a general decrease in the percent crystallinity was observed going from film cast to first press and finally to processing with the hydraulic press at 25° C. or 50° C. This is the same trend observed with blank PCL showing that for a given loading, further processing will always lead to a more imperfect crystal structure and hence changes in amorphous and mesophase content of the polymer. When it comes to the amorphous phase, no clear trend is observed while progressing into the different manufacturing phases for 5% w/w and 20% w/w DTG. At 10% w/w loading, the percent of amorphous content in the polymer increases from 44.06% with the film cast to 56.46% for processed 50° C. samples. For tablets with higher drug loading, despite the extruded flow and increased pressure application for processed samples, the mesophase content may not necessarily increase after the first press. For 5% w/w DTG, the percent of mesophase content for three manufacturing steps with detectable mesophase content is as follows: first press samples had the highest mesophase content with 15.49% followed by processed 25° C. with 14.65% and finally processed 50° C. samples with 14.23%. DTG 10% w/w showed a different trend with processed 25° C. samples having the highest mesophase content (19.76%) followed by first press samples (17.27%) and finally processed 50° C. samples with 15.90% mesophase content. Finally, processed 25° C. samples also had the highest mesophase content (18.95%) for the DTG 20% group, however, processed 50° C. which had the lowest mesophase for the two previous groups displayed higher mesophase content (16.63%) than first press samples (12.10%). Overall, percent crystallinity decreases with further processing for all loading conditions. However, an increasing or decreasing trend was not observed with the amorphous or mesophase content in the polymer for the three loading conditions and each loading condition presented us with a different trend.

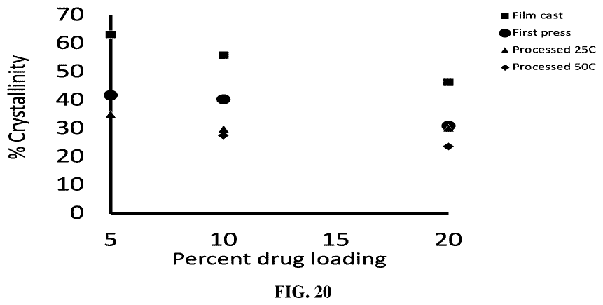

To get more insight into the data, the change in percent crystallinity for the different samples was analyzed as the DTG loading was increased from 5% w/w to 20% w/w (FIG. 20).

It is seen that for each manufacturing step as the amount of drug in each tablet is increased, the percent crystallinity decreases. Without wishing to be bound by theory, it is believed that the drug acts as an impurity and may prevent the crystallization of the PCL polymer chains. This decrease in percent crystallinity will be accounted for by changes in the percent amorphous or mesophase of the polymer. All calculated percent crystallinity values were obtained using only one XRD scan.

ii. Morphological Analysis at Specific Time Points for a Period of 3 Months

Additional morphological analyses of the polymer were performed, to test how the different phases of the polymer change as the samples are immersed in a release medium (PBS) for a period of 3 months at 37° C. Tablets taken out at specific timepoints (24h, 72h, 1 week, 2 weeks, 3 weeks, 1 month, 2 months, 3 months) were analyzed to assess the stability of the polymer morphology over time in the release medium. To assess the variability in the samples, three (3) XRD scans were taken for each sample. Since it was previously assumed that the induction of extruded flow, increased temperature and pressure application will result in the maximum mesophase formation, only DTG tablets with 10% w/w loading processed at 25° C. and 50° C. were analyzed. Blank PCL tablets without the drug were processed similarly and used as controls to determine if the stability of the system changes with the presence of the drug over time. Three spots per tablet were evaluated to account for the uneven dispersion of the drug in the polymeric matrix.

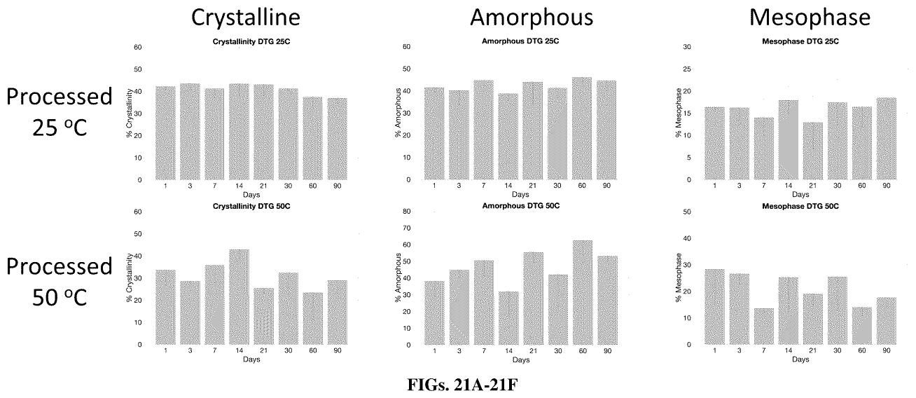

FIGS. 21A-21F show no changes in phase consent for the processed drug loaded samples for the three months of study. Drug loaded PCL tablets showed the same results with all p-values being much higher than 0.01 or 0.05. Hence, all three phases in the polymer are relatively stable at 37° C. in the release medium, and it can be stated that the percent mesophase content calculated at day 0 for all the samples stays constant for a span of 3 months, increasing the system's applicability for long term-controlled release devices.

iii. In Vitro Release Studies

The DTG-loaded PCL tablets were evaluated for their in vitro release kinetics to determine the impact of the processing history and induced morphology on drug release behavior over 90 days. Due to the low solubility of DTG in aqueous solution, the results in this section were assessed with the knowledge that some of the release periods reached saturation. Thus, the results may not have been under sink condition. To account for this, the DTG release studies were repeated (Example 5 below), while maintaining the sink condition. A comparison between the three loading conditions shows a gradual decrease in cumulative percent release of drug from 5% w/w to 20% w/w. This may be in part due to the low saturation of DTG in PBS (approximately 0.05 mg/ml). Typically, in instances where the drug concentration in the tablet is well below the saturation limit, an increase in drug loading would be expected to result in a direct increase in cumulative drug release. However, for all the tested drug loadings (5% w/w, 10% w/w, and 20% w/w), the concentration of DTG in first press and processed tablets is well above the saturation limit. Thus, regardless of the percent drug loading in each tablet, the total mass release is limited due to the low saturation solubility, which acts as a rate limiting step. PCL has a very slow degradation time (2-4 years) and for the entirety of the release studies experiment (3 months), there were no significant signs of tablet erosion or color change observed. This implies that the release of DTG occurred primarily due to diffusion through the PCL matrix. To this end, the in vitro release profiles are believed to represent a time-dependent release based on Fick's first law.

The diffusion flux as a function of time was plotted and calculated at various time points for first press, process 20 C, and processed 50 C for the 5% w/w, 10% w/w, and 20% w/w DTG loadings. For all three loadings and for three processing conditions in each, it was observed that the release rate of DTG is very high in the first 7 days, but drops significantly after 2 weeks of release studies. Beyond the 2-week time point, the rate becomes slower and the diffusive flux approaches a constant.

To better understand the in vitro release profiles, the Higuchi pseudo steady-state model can be used, which describes drug release from thin films containing finely dispersed drug particles into sink conditions (Siepmann, J. & Peppas, N. A. Higuchi equation: Derivation, applications, use and misuse. Int. J. Pharm. 418, 6-12 (2011); Higuchi, T. Rate of Release of Medicaments from Ointment Bases Containing Drugs in Suspension. J. Pharm. Sci. 50, 874-875 (1961)). In this model, the following assumptions are made:
(1) Drug transport through the PCL is rate limiting, whereas drug transport to the release medium from the surface of the tablet is rapid.
(2) The release medium acts like a “perfect sink”: The drug concentration in the surrounding PBS release medium can be considered negligible.
(3) The initial drug concentration in the PCL is much higher than the saturation solubility concentration of the drug in the release medium.
(4) The drug is finely dispersed within the PCL (the size of the drug particles is much smaller than the thickness of the film).
(5) The drug is initially homogeneously distributed throughout the PCL.
(6) The dissolution of drug particles is rapid compared to the diffusion of dissolved drug molecules through the PCL matrix.
(7) The diffusion coefficient of the drug through the PCL matrix is constant and does not depend on time or the position within the film.
(8) Edge effects are negligible: The surface of the PCL exposed to the release medium is large compared to its thickness. The mathematical description of drug diffusion can be restricted to one dimension.
(9) The PCL does not swell or dissolve during drug release.

In the context of the above factors, the initial release of DTG from the matrix delivery system happens at the surface of the tablet through diffusion in the perfect sink conditions of the release medium. Thus, a rapid diffusion flux is observed in the initial first week of release, where the surface concentration of the drug operates at saturation conditions. In the first week where the diffusion flux is very rapid, it is assumed that diffusion through the PCL has minimal effect on the release rate and dissolution of the drug from the surface of the delivery system is the governing step. Only when all drug particles located in the region next to the surface are finally dissolved does the concentration of dissolved drug molecules at the surface fall below saturation concentration of 0.05 mg/ml. At this point, based on an inward moving concentration gradient, dissolved drug molecules located further away from the surface begin to diffuse through the tablet matrix into the release medium. For simplification of the release kinetics, the Higuchi model assumes pseudo steady-state behavior in which the concentration gradient remains at saturation concentration (0.05 mg/ml) within the PCL at the moving diffusion front (due to rapid dissolution) and Omg/ml at the surface (due to rapid release to sink conditions). Hence there is an initial rapid release, followed by a constant diffusion flux. This can be explained by the constant dC of the pseudo steady state gradient, and the small dx of the PCL thickness, with the moving diffusion front having minimal effect on the gradient.

In the in vitro release study, values were averaged from the set of triplicates (n=3) for each of the processing conditions with each loading having in total nine (9) tested tablets for drug release. Error bars calculated are very narrow which indicate that for each processing condition at the three tested loadings, there is reproducibility, less dispersion and the absence of major outliers. Nonetheless, in the next section, additional experiments were performed to assess the reproducibility of the release profiles. Different trends between the processing conditions for the three drug loadings were observed. For 5% w/w DTG, processed 50° C. tablets release the fastest with almost 94.93% of the drug released after 3 months. Processed 25° C. and first press tablets release much slower with, respectively, 45.18% and 29.94% of the drug released after 90 days. DTG 10% w/w have a similar trend with processed 50° C. tablets releasing the fastest after 3 months (67.44%), however, processed 25° C. show a slower release rate than first press tablets (36.92% vs 44.07%). Finally, DTG 20% w/w show that first press tablets release faster (39.04%) than any of the two processed tablets with processed 25° C. samples now releasing the slowest (34.78%) after 3 months.

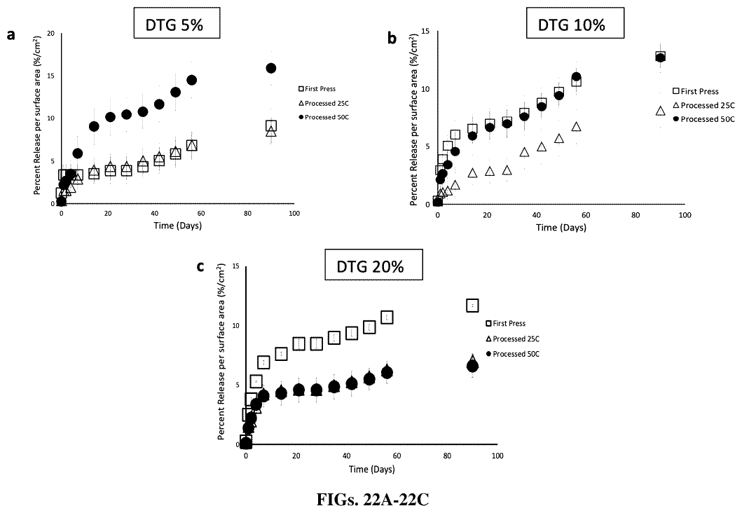

Samples across the different processing conditions did not have the same surface area. As such, FIGS. 22A-22C show the percent release per surface area curves where the cumulative percent release curves are normalized by the surface area of tablets in the different processing conditions. Typically, as the surface area to volume ratio of a delivery system is increased, we expect to see an increase in the cumulative release (i.e., more surface drug contact with the release medium should be represented by higher rates of release). However, in the release studies that did not control for surface area, the change in surface area does not appear to have a significant impact on cumulative release. Indeed, processed 25° C. and processed 50° C. tablets have higher surface area than the first press tablets. However, these processed tablets are not always releasing the fastest for all the drug loading cases (i.e., processed 25° C. have the smallest cumulative percent release for 10% w/w and 20% w/w DTG loading observed in the in vitro release data described above). FIGS. 22A-22C show the cumulative percent release per surface area for all three loadings. These graphs were obtained using the cumulative percentages from the studies that did not control for surface area and normalizing by the surface area of the samples. In FIGS. 22A-22C, processed tablets now show slower percent release per surface area than first press tablets (FIG. 22C). However, in the cases of DTG 5% w/w (FIG. 22A) and DTG 10% w/w (FIG. 22B), processed 50° C. samples are now showing the fastest release of drug per surface area after 3 months. Hence, differences in surface area cannot be the only factor to explain the differences in release as it cannot be concluded that an increase in surface area always leads to faster release of the drug. With the normalization performed in FIGS. 22A-22C, the results be interpreted using Higuchi pseudo steady state model to calculate the diffusion coefficient of the drug in the three different processing conditions for 5% w/w, 10% w/w, and 20% w/w loading of DTG. The fit (R²) between the Higuchi modeled release data and the experimental data were mostly above 0.90, except for one fit with an R² of 0.77.

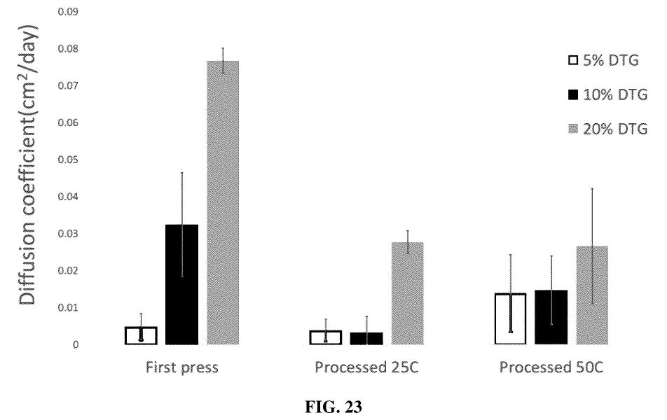

Diffusion coefficients were calculated for each processing condition for all three loading conditions (FIG. 23). These calculated diffusion coefficients help bring more clarity to the release curves shown in FIGS. 22A-22C. Indeed, for the 5% w/w loading case, processed 50° C. tablets showed the highest diffusion coefficient (0.0139 cm²/day), while first press and processed 25° C. had similar diffusion coefficients with 0.0049 cm²/day and 0.0038 cm²/day, respectively. This trend in diffusion coefficient explains the fast release observed for processed 50° C., while first press and processed 25° C. showed much slower release of DTG over the 90-day release study experiments (FIGS. 22A-22C). 10% w/w and 20% w/w DTG groups showed the same trends as the samples with the highest diffusion coefficients showed the fastest release of drug. Finally, it appears that the first press samples show concentration dependent diffusion coefficient as the release seems to increase with increasing drug loading.

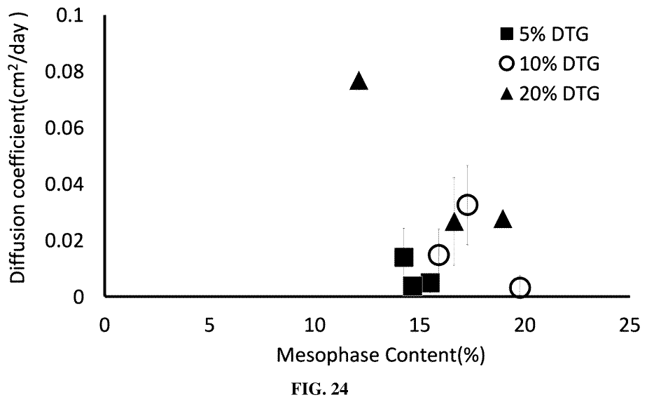

FIG. 24 displays the correlation between mesophase content and diffusion coefficient for all three loading conditions. This figure helps to elucidate the relationship between the calculated diffusion coefficients and the PCL morphology, specifically the mesophase content of the polymer.

As shown through these morphological study experiments, the mesophase content along with the other phases (crystalline and amorphous) of PCL remain stable during the three months of release studies. Hence, the mesophase content calculated for samples at each processing condition was used for all three loadings at day 0 (FIG. 22). Overall, for all three loading conditions, a negative correlation was observed between the mesophase content of PCL and the diffusion coefficient. For DTG 5% w/w and DTG 10% w/w, the calculated correlation coefficients were respectively −0.68 and −0.54 which suggests that there is a moderate negative correlation. 20% w/w DTG-loaded tablets showed the strongest negative correlation with a correlation coefficient of −0.94. Thus, it is seen that first press samples which had the lowest mesophase content (12.10%) at day 0 among the three processing conditions in the DTG 20% w/w group had the highest diffusion coefficient (0.0768 cm²/day), whereas processed 25° C. which showed the highest mesophase content (18.95%) showed the lowest diffusion coefficient (0.0277 cm²/day) and slowest release of drug.

DTG is believed to diffuse through the amorphous regions of the PCL network as the highly ordered crystalline regions would not allow penetration of the drug. Thus, an increase in density or decrease in free volume of the amorphous region of the network would be expected to result in a reduction of DTG diffusion, as this would result in a direct decrease in the available area for the DTG to travel through. Previous work shows that at low processing pressures, slight plastic crystal morphology could be observed, which formed primarily at the expense of the crystalline phase (Baptista, C. et al. The effect of temperature and pressure on polycaprolactone morphology. Polymer 191, 122227 (2020)). However, upon increasing the processing pressure (i.e., 10,000 lbs) and plastic deformation, mesophase formation was seen to form at the expense of both the amorphous and crystalline phases due to the connection between all these phases of the polymer. This mechanism of formation of the mesophase in PCL may explain the results observed in FIG. 17, with the implication that the lower the diffusion coefficient observed is directly related to the decrease in free volume of the amorphous phase contribution to the plastic crystal mesophase formation.

In conclusion, the present study demonstrated the ability to achieve sustained release of DTG from biodegradable PCL tablets at loadings of 5% w/w, 10% w/w, and 20% w/w for a period of 3 months. The release of DTG appeared to primarily follow Fickian diffusion, with negligible contributions from PCL degradation. This release behavior was successfully modeled using the Higuchi equation, suggesting the tablets behave as a pseudo steady-state system with finely dispersed drug particles. To this end, it was shown that to successfully model each sample using the Higuchi equation, a diffusion coefficient for each of the different processing conditions (first press, processed 25° C., and processed 50° C.) had to be calculated. This variable diffusion coefficient was primordial in providing an explanation for the observed differences in cumulative percent per surface area. Analysis by XRD showed the presence of the plastic crystal mesophase in all DTG loaded tablets. Using peak deconvolution of the integrated XRD profiles, the mesophase content of all the samples and found trends between processing conditions was quantified based on the drug loading. Nonetheless, it was demonstrated that with increasing mesophase content in each drug loaded PCL sample, there was a decreasing diffusion coefficient. This negative correlation was particularly strong for DTG 20% w/w tablets with an R² value of −0.94. The variable diffusion coefficient observed between samples processed under varying conditions is thought to relate to the decrease in free volume associated with mesophase formation. These findings indicate that mesophase formation may impact the long-acting release behavior and may serve as a means for controlling the rate of drug diffusion.

### Example 4. Preparation of Melt Cast and Melt Cast-Induced Mesophases of Dexamethasone-, Meloxicam-, and Dolutegravir-Loaded Polycaprolactone Tablets

This section presents an alternative method to induce the mesophase into drug-loaded PCL tablets, wherein the initial sample is first prepared by melt casting. The aim of this example was to first demonstrate the absence of mesophase in the melt cast samples, and then to induce the mesophase via processing. This study design allows for a direct comparison of drug diffusion between similar samples with and without mesophase. The release profiles in this study were conducted under sink conditions.

The morphology is analyzed and then the inducement of the mesophase is formed. The importance of this section is that many traditional fabrications are based on solvent film casting, and if pressure is not applied, a mesophase is not obtained. This method also eliminates a feature referred to as an “accordion structure” of the first press presented in the previous examples (Example 1, 2, and 3). This “accordion structure” was seen as the multiple layers of the folded film cast, which did not fuse together upon tablet formation. The presence of these layers in the “accordion structure” may introduce errors in the release per surface area calculations (as the total surface area is higher than that of the tablet). Utilizing the melt case method in this section, controls could be obtained with true “no mesophase” morphology and the sample surface area calculations could be made more accurate. This method is also more relevant to some industrial applications of making delivery system such as melt extrusion.

### Materials and Methods

i. Tablet Preparation

Polycaprolactone tablets were loaded with 10% w/w DEX, MLX, and DTG by first solubilizing approximately 225 mg of PCL and 25 mg of drug in dichloromethane (2 mL) and film casting on a glass Pyrex dish. Upon evaporation of DCM, the thin casted film was pressed in a pellet die at 10,000 lbs for 15 minutes. This step is performed to mix the drug and polymer, although it can be avoided. The resulting pellet was then melt casted in a silicone mold at approximately 65° C. to allow for fusion of the pellet layers. This melt casted sample served as the “melt cast-control” for the experiment. Select samples were further processed at 20,000 lbs at 50° C. for 15 minutes on a Carver Autopress to induce mesophase morphology.

ii. Release Studies

Each tablet was placed in a scintillation vial containing 20 mL of PBS to mimic sink conditions in a physiological environment. Release vials were incubated at 37° C. for the entirety of the release experiment. Periodically, the PBS solutions for each sample was collected and analyzed via UV spectroscopy to quantify the amount of drug released. Following the collection of PBS solutions, the release medium for each tablet was subsequently changed with fresh PBS and then incubated again until the next time point. Drug concentrations were quantified using the absorbance values obtained from the SoftMax PRO software 4.3.1 of the Spectra max 384 Plus Ultraviolet Spectrophotometer. Using the absorbance values and the calibration curves, concentrations were calculated for the different time points of the release studies. Solution concentrations which fell outside the linearity region were diluted and new absorbance values were obtained, and concentrations were once again calculated using the appropriate dilution factor.

iii. XRD Phase Content Calculation

XRD was performed using a Bruker D-8 Advance X-Ray Powder Diffraction system running Da Vinci Software and a Cu X-Ray tube operating at 40 kV and 40 mA (1600W) equipped with a Bruker Vantec-500 Xe—CO2 gas filled detector with a 13.5 cm diameter window set at 20 cm from the goniometer center. System wavelength was 0.15418 nm. The system optics composed of a polycap with an output beam divergence of 0.25° for Cu and a spot diameter of less than 4.0 mm and a 79-mm long 0.3-mm collimator on the incident beam path. Scans were run at 20 angle from 20° to 80° with a step size of 15° and a counting timestep of 60s (300s total). XRD scans are taken at 3 different spots in each tablet to display a more accurate picture of the polymer morphology and dispersion of the drug in the delivery system.

The phase content of each sample was calculated from the obtained XRD scattering profiles by peak deconvolution using the Fityk v1.3.1 software. The fitting method utilized Gaussian profiles for all scattering peaks and halos. As such, the fitting involved the use of 5 peaks to characterize the major PCL peaks occurring approximately at 20 values of 21.4° and 23.8°. Other PCL peaks were not included in the analysis as those can greatly vary in intensity depending on the resolution of the apparatus. The first peak was used to characterize the broad amorphous. Two peaks were then added to represent the (110) and (200) orthorhombic crystalline face peaks. Finally, two mesophase peaks were added to fit a computed model with the experimental data with an R² value of at least 0.97. The content of each phase (amorphous, crystalline, and mesophase) was calculated by the area under the diffraction peaks, using the ratio from one phase to the total scattering peaks.

### Results

i. Tablet Preparation

The compositions of various tablets produced are shown in the following table.

TABLE 1
Compositions of tables produced from the melt cast method
Dexamethasone (10% Loading)

| | | | | | Surface | Drug area | |
|---|---|---|---|---|---|---|---|
| | PCL | DEX | Radius | Thickness | area | density | Processing |
| | (mg) | (mg) | (cm) | (cm) | (cm\hat{ }2) | (mg/cm\hat{ }2) | Method |
| Tablet 1 | 239.8 | 24.9 | 1.176 | 0.056 | 9.103279935 | 2.735277853 | Processed |
| | | | | | | | at 50 C., |
| | | | | | | | 20000 lbf |
| Tablet 2 | 223.4 | 25.4 | 1.427 | 0.031 | 13.07258172 | 1.942997989 | Processed |
| | | | | | | | at 50 C., |
| | | | | | | | 20000 lbf |
| Tablet 3 | 232.7 | 25.5 | 1.1545 | 0.053 | 8.759129455 | 2.911248216 | Processed |
| | | | | | | | at 50 C., |
| | | | | | | | 20000 lbf |
| Tablet 4 | 228.9 | 24.8 | 0.7265 | 0.17 | 4.092284144 | 6.060185248 | Control |
| | | | | | | | Melt Cast |
| Tablet 5 | 232.8 | 24.2 | 0.766 | 0.248 | 4.880300825 | 4.958710717 | Control |
| | | | | | | | Melt Cast |
| Tablet 6 | 218.7 | 25.2 | 0.808 | 0.166 | 4.944816571 | 5.096245662 | Control |
| | | | | | | | Melt Cast |

Meloxicam (10% Loading)

| | | | | | Surface | Drug area | |
|---|---|---|---|---|---|---|---|
| | PCL | MLX | Radius | Thickness | area | density | Processing |
| | (mg) | (mg) | (cm) | (cm) | (cm\hat{ }2) | (mg/cm\hat{ }2) | Method |
| Tablet 1 | 213.9 | 24.8 | 1.2095 | 0.063 | 9.67037982 | 2.564532155 | Processed |
| | | | | | | | at 50 C., |
| | | | | | | | 20000 lbf |
| Tablet 2 | 214.4 | 25.8 | 1.1055 | 0.062 | 8.109526634 | 3.181443401 | Processed |
| | | | | | | | at 50 C., |
| | | | | | | | 20000 lbf |
| Tablet 3 | 236 | 25.5 | 1.257 | 0.046 | 10.291047 | 2.477881988 | Processed |
| | | | | | | | at 50 C., |
| | | | | | | | 20000 lbf |
| Tablet 4 | 221.6 | 25.1 | 0.72 | 0.217 | 4.238888136 | 5.921364093 | Control |
| | | | | | | | Melt Cast |
| Tablet 5 | 225.7 | 25.8 | 0.692 | 0.214 | 3.939255595 | 6.549460775 | Control |
| | | | | | | | Melt Cast |
| Tablet 6 | 225.1 | 25.6 | 0.7175 | 0.183 | 4.059621005 | 6.306007376 | Control |
| | | | | | | | Melt Cast |

Dolutegravir (10% Loading)

| | | | | | Surface | Drug area | |
|---|---|---|---|---|---|---|---|
| | PCL | DTG | Radius | Thickness | area | density | Processing |
| | (mg) | (mg) | (cm) | (cm) | (cm\hat{ }2) | (mg/cm\hat{ }2) | Method |
| Tablet 1 | 227.7 | 24.5 | 1.1845 | 0.05 | 9.187683534 | 2.666613397 | Processed |
| | | | | | | | at 50 C., |
| | | | | | | | 20000 lbf |
| Tablet 2 | 218.6 | 25.3 | 1.0805 | 0.069 | 7.803934492 | 3.24195443 | Processed |
| | | | | | | | at 50 C., |
| | | | | | | | 20000 lbf |
| Tablet 3 | 228.1 | 25.4 | 1.103 | 0.058 | 8.04614029 | 3.156793081 | Processed |
| | | | | | | | at 50 C., |
| | | | | | | | 20000 lbf |
| Tablet 4 | 224.9 | 24.7 | 0.8145 | 0.149 | 4.930860046 | 5.009268113 | Control |
| | | | | | | | Melt Cast |
| Tablet 5 | 222.4 | 26.2 | 0.788 | 0.186 | 4.822420121 | 5.43295676 | Control |
| | | | | | | | Melt Cast |
| Tablet 6 | 222.7 | 25.5 | 0.796 | 0.144 | 4.701330574 | 5.423996377 | Control |
| | | | | | | | Melt Cast |

ii. XRD Phase Content Calculation

As can be seen in Table 2, the melt cast PCL sample had zero mesophase while the processed PCL samples had varying degrees of mesophase with all three drug loadings.

TABLE 2
XRD phase content calculation

| | Phase Content (%) | | |
|---|---|---|---|
| | Crystalline | Amorphous | Mesophase |
| Melt Cast Control | 59.85036 | 40.14964 | 0 |
| DTG Processed Tablet | 31.00385 | 31.64591 | 37.35024 |
| MLX Processed Tablet | 34.08205 | 44.51078 | 21.40717 |
| DEX Processed Tablet | 39.41758 | 27.65021 | 32.93221 |

iii. Release Studies

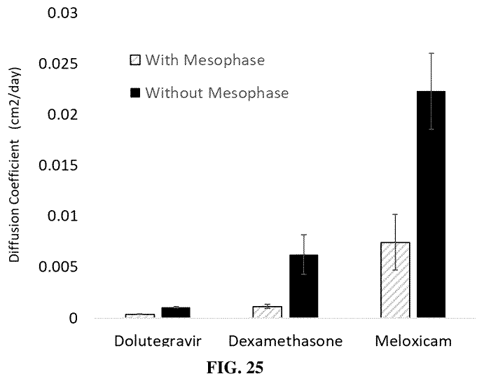

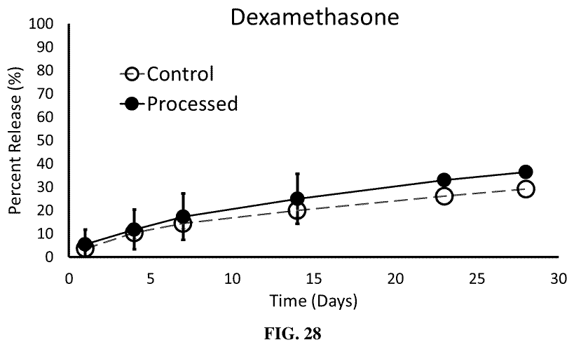

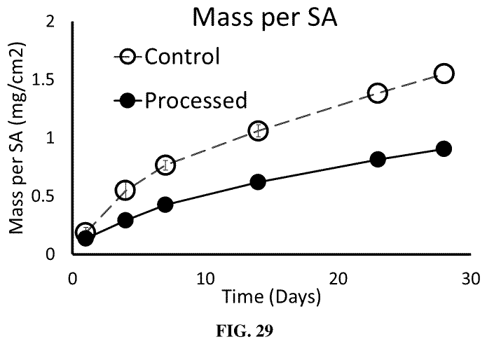

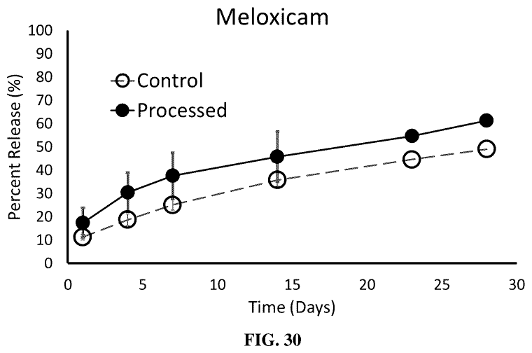

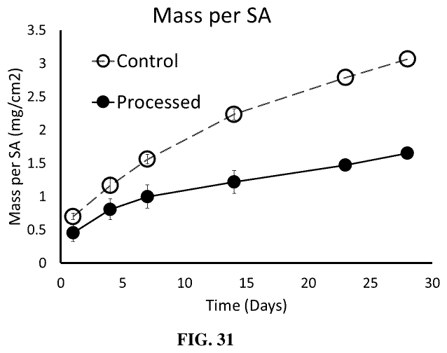

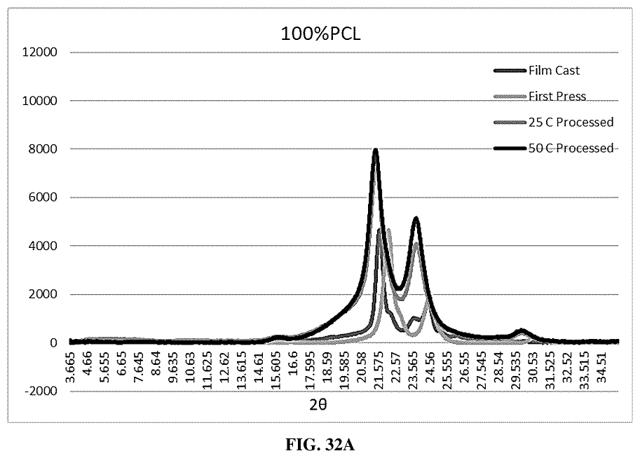

The 10% DTG, MLX, and DEX loaded PCL tablets were evaluated for their in vitro release kinetics to determine the impact of the processing history and induced morphology on the drug release behavior (FIGS. 25-31). Independent of processing conditions and drug loading, all PCL matrices showed sustained release over the 30-day duration of the experiment.

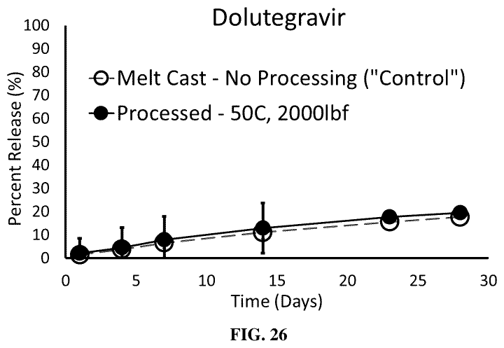

While PCL is subject to hydrolytic degradation, its degradation times can be very slow (on the order of 2-4 years) due to its hydrophobic nature. In this study, there were no significant signs of tablet erosion observed over the duration of 30 days. This shows that the release of each drug occurred primarily due to diffusion through the PCL matrix. To this end, the release profiles presented in FIGS. 26-31 are considered to represent a time-dependent release based on Fick's law.

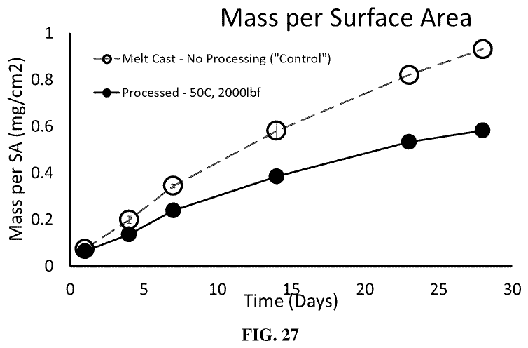

While the total cumulative release between the samples with and without mesophase appear similar for each drug loading (FIGS. 26, 28, and 30), an important attribute to note is the difference in the release per surface area of each sample (FIGS. 27, 29, and 31). A constraint in this analysis between the different processing conditions is the inherent change in surface area associated with each processing condition. Typically, increased surface area to volume ratio of a release device is expected to result in an increase in the cumulative release (i.e., more surface drug contact with the release medium should be represented by higher rates of release). However, in the release demonstrated here, the change in surface area does not appear to have a significant impact on cumulative release. Rather, this is curiously associated with a decrease in percent release per surface area. To further demonstrate this difference, the diffusion coefficient was approximated using the Higuchi equation as described above (FIG. 25) demonstration reduced diffusion coefficient as mesophase is induced.

### Example 5. Preparation of Blank and Curcumin (CUR)-Loaded PCL Films

### Materials and Methods

i. Sample Preparation

In this example, curcumin was utilized as a model drug. For sample preparation, the polymer and the drug were first dissolved in chloroform (CHCl₃, Sigma), curcumin (Sigma, 97% purity), and CAPA 640 PCL (Perstorp Holding AB, Molecular Weight: 37 kg/mol). All samples, including blanks, weighed 250 mg. Loaded samples were prepared with varying amounts of curcumin (the drug), at concentrations of 1%, 5%, 10%, 30%, and 50% of the total mass.

Each sample was prepared by weighing the respective amounts of polymer and drug, then dissolving them in 2 ml of chloroform. Complete dissolution required three rounds of sonication and vortexing. After complete dissolution, this mixture was transferred into a petri dish (62 mm in diameter), and films were cast by allowing the solvent to evaporate. After 24 hours, the film was carefully removed from the dish.

ii. Processing of Curcumin and Blank PCL Films

The films produced in the previous section were processed using a hand press (Specac Inc) and a 20 mm die. These films were pressed for 5 minutes at room temperature under 10,000 lbs of pressure. At the end of this process, all the films had transformed into cylindrical tablets. In this stage, nine distinct tablets were produced for each of the different loadings. Under these pressing conditions, three tablets were set aside for preservation.

The remaining six tablets for each loading were further processed using a Carver Auto Series Hydraulic Press equipped with a heating/cooling system (Carver, Wabash, Indiana). Three of these six tablets were processed at 25° C.; they were placed on a stainless-steel plate and then compressed for 10 minutes without engaging any pressure, in order to elevate the sample temperatures. This step was followed by applying 10,000 lbs. of pressure for 5 minutes and immediate cooling while maintaining the pressure. This stage is critical as it helps retain the morphological alterations induced by pressure and heating. The same procedure was repeated for the remaining three tablets, with the exception that the procedure was performed at 50° C. The dimensions of the tablets were measured upon completion of the pressing process.

### Results

i. XRD Analysis of Curcumin and PCL Mesophase Formation

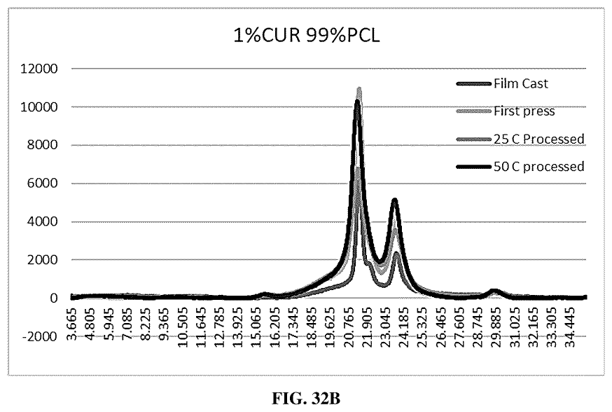

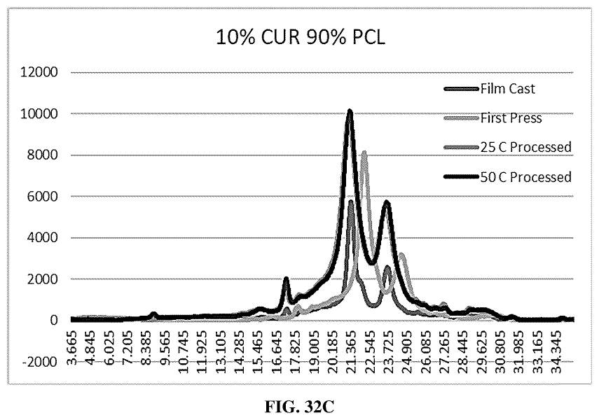

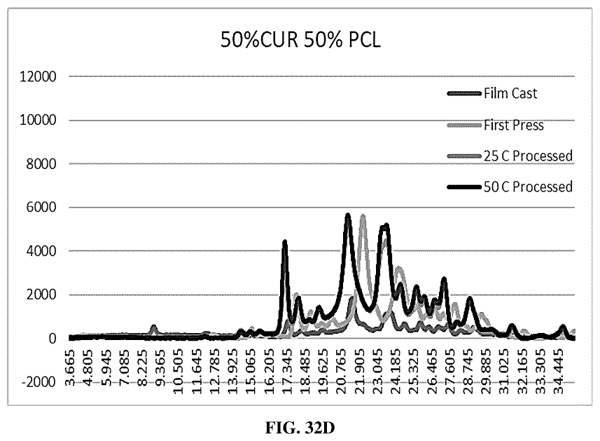

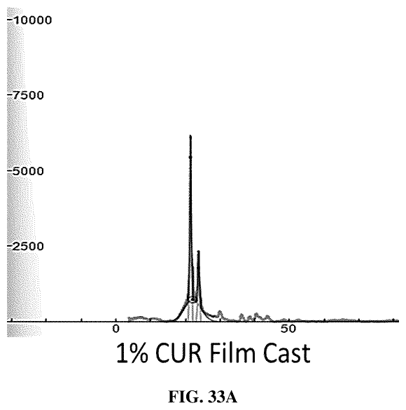

An integrated XRD profile of pure PCL revealed broadening of the peak when processed at 25° C. and 50° C. compared to the initial pressing and film-cast samples (FIG. 32A). This expanded space signifies the induction of the mesophase. A similar trend was observed with the drug loaded films as seen in FIGS. 32B-32D. This occurrence was independent of the drug loading in the system.

XRD scans were conducted at room temperature. 2D azimuthal XRD profile of different process film cast, first press, 25° C. press, 50° C. press across various drug concentrations were generated. The increase in the intensity of the Debye-Sherrer rings seen in the 25° C. press, 50° C. press around the equator indicated induction of plastic mesophase crystals (Baptista, et al., Polymer. 191: 122227 (2020)). The results indicated that the film-cast samples exhibited an isotropic profile, characterized by a diffused halo and a sharp crystalline peak (data not shown). The amorphous phase of the film-cast samples demonstrated no conformational order or orientation, hence the weak ring. However, the crystalline peak is more ordered (Wunderlich, Thermochimica Acta, 340-341:37-52 (1999)). The film cast exhibits a diffused amorphous halo and sharp crystalline peaks, which are distinguished by an anisotropic scattering profile, according to Wunderlich's descriptions of orientational, positional, and conformational order (Wunderlich, Thermochimica Acta, 340-341:37-52 (1999)).

In comparison to the processed samples (first press), there was observed peak broadening relative to film-cast samples. This broadening (data not shown) was indicated by the thickening of the Debye-Sherrer rings compared to the film-cast. The peak broadening could be attributed to a shift in structural order from amorphous to a more organized form.

At a low processing temperature of 25° C., the peaks became broader, signifying more changes in the conformational order, and a more intense ring was seen at the equator in the Debye-Sherrer ring compared to the first press. This trend was also evident when the samples are processed at a higher temperature of 50° C. In this case, peak broadening was observed along with the appearance of thick arcs in the Debye-Sherrer ring, which were brighter at the center. The morphology observed in these processed samples is consistent with literature descriptions of mesophase (Wunderlich, Thermochimica Acta, 340-341:37-52 (1999)).

The same trends were noticed across all the PCL systems, regardless of different drug loadings. In the Debye-Sherrer rings, new crystalline rings began to appear at the 30% Curcumin loading, and more of these rings were visible at 50% drug loading. This could be due to drug saturation in the matrix and the phase separation of the drug and the polymer.

The structural characteristics observed in this PCL-based system resemble those of the plastic crystal mesophase. Plastic crystal mesophases form due to crystallite deformation and slip lines within the molecular framework. These crystals maintain positional order while in a “plastic” state, with disorder in the conformational and angular orders (Androsch, et al., Polymer, 51:4639-4662 (2010)). PCL's long-chain hydrocarbon structure allows for possible orthorhombic structural dislocations and numerous screw and edge dislocations on either side of the methyl groups (Rezgui, F., et al., Polymer 46:7370-7385 (2005)). This causes the individual crystal layers to slide and shift further. The unevenly attached carbonyl groups along the chain orientation can also contribute to the disordering of the crystal and the subsequent creation of the plastic crystal mesophase. Condis crystal mesophases differ from plastic mesophases in that they exhibit partial or total conformational chaos, with birefringent characteristics linked to positional and orientational order. A condis crystal mesophase forms at a temperature between the onset and end of the PCL melt, which is between 50 and 60° Celsius (Baptista, et al., Polymer. 191: 122227 (2020)).

ii. Peak Deconvolution and Calculation of % Mesophase Induction

Deconvolution analysis was conducted on the curcumin-loaded PCL systems to quantitatively assess the morphology of the system. The Gaussian profiles proposed by Stoclet et al. for all dispersed peaks and halos were adopted to account for the crystalline, amorphous, and mesophase contents (Stoclet, et al., Macromolecules 43:7228-7237 (2010)). The film-cast sample was taken as the control for this calculation.

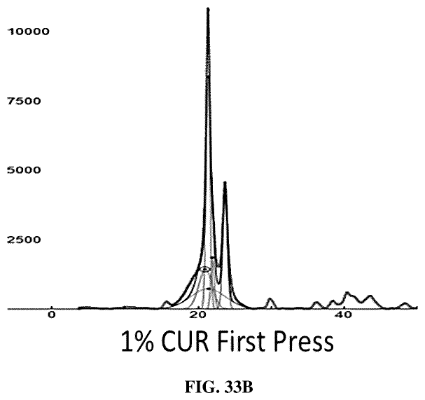

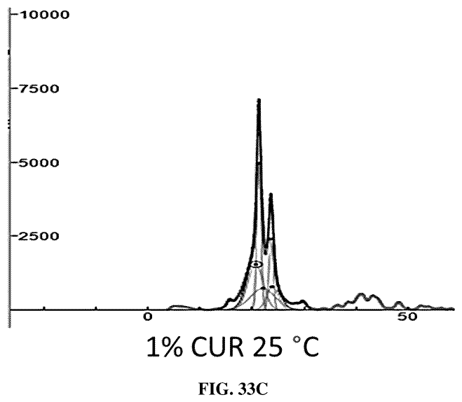

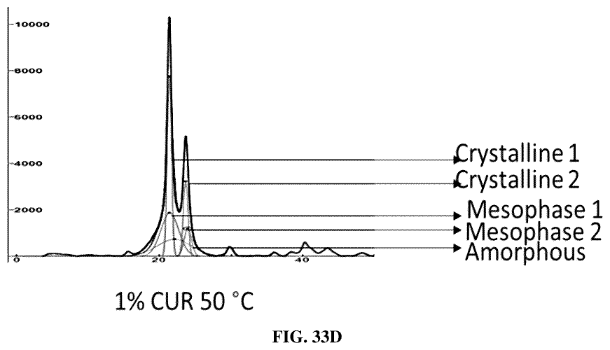

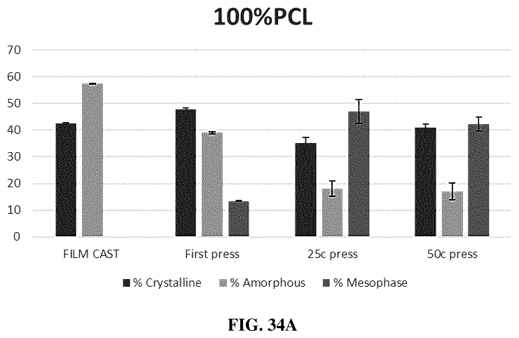

To calculate the crystalline, amorphous, and mesophase peaks, the ratio between the total scattering area and the total peak area was analyzed. These peaks were characterized, and curve fitting was performed, requiring an R² value of more than 0.97. Based on FIGS. 33A-33D, a pattern can be discerned in the calculation of mesophase content for PCL systems loaded with 1% curcumin. A higher percentage of mesophase is associated with pressure, temperature, and plastic deformation. An R² value greater than 0.97 was achieved when analyzing film-cast samples without adding mesophase peaks (Gaussian peaks), suggesting that the film-cast samples displayed a characteristic semi-crystalline morphology without any mesophase content.

The analysis revealed a rise of 33.58% in mesophase morphology from the first press to the processed sample of 100% PCL, with a corresponding reduction of 20.92% in the amorphous content of the system. There was an increase of 11% in the mesophase morphology observed between the first press and the processed sample of 50% PCL and 50% Curcumin, with a concurrent decrease of 4.39% in the amorphous content in the system.

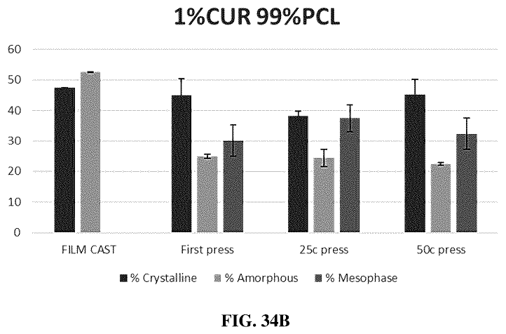

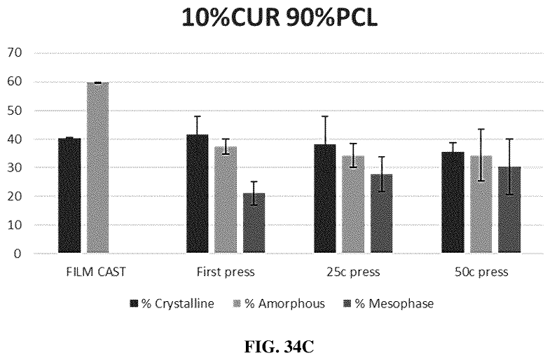

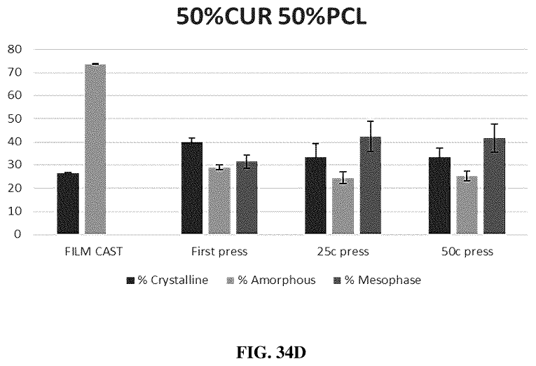

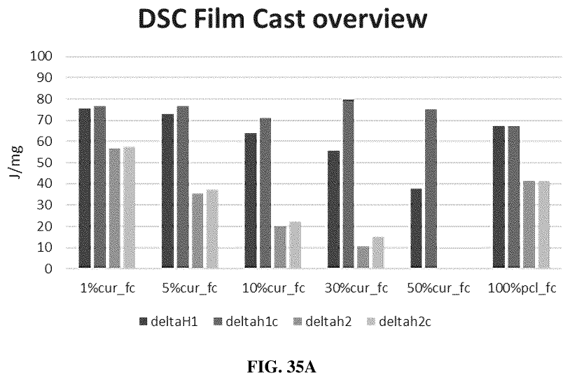

A similar trend was observed in all the drug loading of PCL-Curcumin systems. This discovery that the mesophase forms as a consequence of plastic deformation and crystal disorganization may be explained by the previously described process. Increasing the force and temperature is consistently associated with a rise in mesophase formation, even when the mesophase variation between 1%, 10%, and 50% drug loading is disregarded. The results are shown in FIGS. 34A-34D.

iii. Thermal Analysis by DSC

The thermal behavior of both processed and unprocessed PCL-Curcumin systems was studied using PerkinElmer's MDSC, considering the processing temperature, drug loading, and pressure. The samples were analyzed using the following protocol: (1) the initial temperature was set to 0° C.; (2) the temperature was held at 0° C. for 1 minute; (3) the temperature was heated from 0° C. to 200° C. at a rate of 10° C./min; (4) the temperature was held at 200° C. for 1 minute; (5) the temperature was cooled from 200° C. to 0° C. at a rate of 10° C./min.; (6) the temperature was held at 0° C. for 1 minute; (7) the temperature was heated from 0° C. to 200° C. at a rate of 10° C./min; and (8) the temperature was reduced to room temperature before opening the furnace. The samples were heated from 0° C. to 200° C. because the melting temperature of PCL is 60° C. (Nair, et al., Current Developments in Biotechnology and Bioengineering in Production, Isolation and Purification of Industrial Products, pages 739-755 (2017)), and the melting temperature of curcumin is 184° C. (Kunnumakkara, et al., ACS Pharmacology & Translational Science, 6 (4): 447-518).

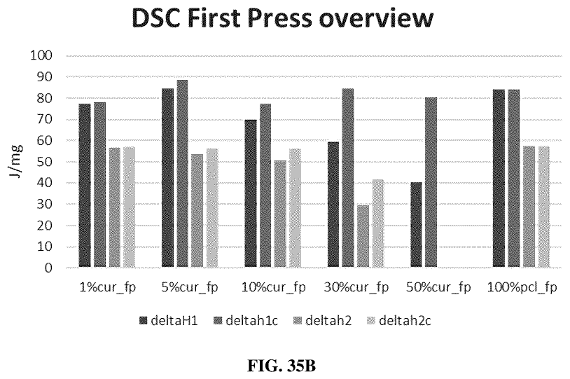

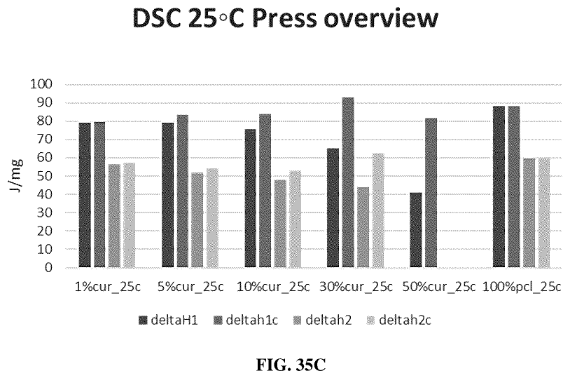

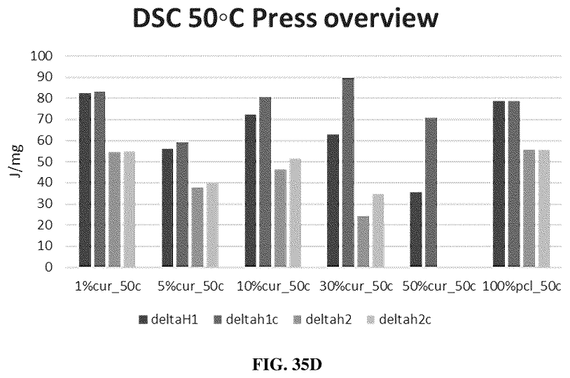

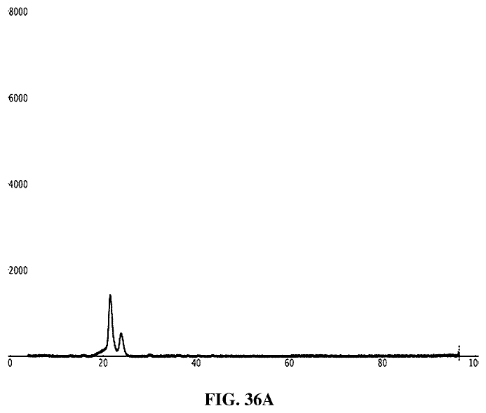

As shown in FIGS. 35A-35D, a general trend can be observed. In the first heat run, the delta H1 (first heating) and the deltahlc (first cooling) value remains relatively similar, ranging between 70-90 J/mg across different processing conditions and drug loading percentages. However, a different trend is observed in the second heat curve (delta H2) and the second cooling curve (delta H2c).

Interestingly, in FIG. 35A, for the film cast, a linear decrease of the delta H2 and deltah2c value was observed as the drug loading percentage increases. From the previous sections, it was established that in the film cast system no mesophase content was observed. A similar trend was observed in FIG. 35B (first press) samples, but in this process, the values do not decrease linearly.

Surprisingly, FIGS. 35C and 35D, which represent samples pressed with heating at 25.° C. and 50° C. respectively, show that the second run delta values were more similar.in all processing conditions. However when observing the sample with drug loading of 50% PCL-50% Curcumin, it was noted that the second heat run was missing. During the first run, the sample was heated until all components melted, at this point the polymer and the drug could interact with each other r at the molecular level, thereby inhibiting the polymer from recrystallizing thus sowing no melting

A similar trend is observed with the 30% curcumin system. Hence, slight recrystallization of the polymer is observed in the second run, but only in the film cast samples. In the other processed samples, an increase in recrystallization was seen in the second run. Table 3 summarizes in more detail the thermal analysis for this section.

TABLE 3
DSC Calculation

| sample | weight | deltaH1 | deltah1c | area1 | peak1 | deltah2 | deltah2c | area2 | peak2 |
|---|---|---|---|---|---|---|---|---|---|
| 1%cur_fc | 3.03 | 75.7389 | [symbol] | [symbol] | 63.8 | 56.9738 | [symbol] | 190.2925 | 57.46 |
| 1%cur_fp | 3.34 | [symbol] | [symbol] | [symbol] | 63.83 | 55.5378 | [symbol] | 189.1702 | 57.46 |
| 1%cur_25c | 2.432 | 78.8439 | [symbol] | 191.7484 | 63.98 | [symbol] | 57.17131 | [symbol] | 58.24 |
| 1%cur_50c | 3.394 | 82.2384 | [symbol] | [symbol] | [symbol] | [symbol] | [symbol] | 184.8447 | 58.1 |
| 5%cur_fc | [symbol] | [symbol] | [symbol] | [symbol] | [symbol] | [symbol] | [symbol] | 79.911 | [symbol] |
| 5%cur_fp | [symbol] | [symbol] | [symbol] | [symbol] | 65.41 | 53.7006 | [symbol] | 195.846 | 57.43 |
| 5%cur_25c | 2.994 | 78.992 | [symbol] | 230.2185 | [symbol] | [symbol] | [symbol] | 5.795 | [symbol] |
| 5%cur_50c | [symbol] | [symbol] | [symbol] | 204.2529 | [symbol] | [symbol] | [symbol] | 137.229 | [symbol] |
| 10%cur_fc | 2.8 | [symbol] | 70.93056 | 178.7449 | [symbol] | [symbol] | 22.20744 | 55.9627 | 54.7 |
| 10%cur_fp | 1.745 | [symbol] | 77.51211 | 121.7328 | [symbol] | 50.8877 | [symbol] | 88.7991 | [symbol] |
| 10%cur_25c | 2.042 | [symbol] | 83.852 | 154.1031 | [symbol] | 47.7819 | [symbol] | 97.5707 | 54.56 |
| 10%cur_50c | 2.078 | [symbol] | 80.41 | 150.3828 | 63.08 | [symbol] | 51.44911 | 96.2202 | 55.4 |
| 30%cur_fc | 3.53 | [symbol] | [symbol] | 196.6479 | [symbol] | 10.7092 | 15.29886 | 37.8036 | 47.77 |
| 30%cur_fp | 2.955 | 59.3177 | 84.73957 | 175.2838 | [symbol] | 29.251 | 41.78714 | [symbol] | 48.58 |
| 30%cur_25c | [symbol] | 65.043 | 92.91857 | 187.4539 | [symbol] | 43.9508 | [symbol] | [symbol] | 49.02 |
| 30%cur_50c | 2.285 | [symbol] | [symbol] | [symbol] | 62.7 | 24.375 | 34.82143 | [symbol] | 48.32 |
| 50%cur_fc | 2.16 | 37.6564 | 75.3128 | 81.3778 | [symbol] | 0 | 0 | 0 | 0 |
| 50%cur_fp | 3.55 | 40.1855 | 80.371 | 126.1825 | [symbol] | 0 | 0 | 0 | 0 |
| 50%cur_25c | 3.79 | [symbol] | [symbol] 1.4472 | [symbol] | [symbol] | 0 | 0 | 0 | 0 |
| 50%cur_50c | 2.355 | 35.398 | [symbol] | [symbol] | [symbol] | 0 | 0 | 0 | 0 |
| 100%pcl_fc | 2.11 | [symbol] | [symbol] | [symbol] | [symbol] | [symbol] | 41.5189 | [symbol] | 56.68 |
| 100%pcl_fp | 2.87 | [symbol] | 84.3038 | 242.7107 | [symbol] | 57.5454 | 57.5454 | 165.6733 | 57.43 |
| 100%pcl_25c | 3.15 | 88.1326 | 88.1326 | [symbol] | [symbol] | [symbol] | [symbol] | [symbol] | 57.92 |
| 100%pcl_50c | 2.648 | [symbol] | [symbol] | [symbol] | [symbol] | [symbol] | [symbol] | 175.3028 | 58.03 |
| Pure curcumin | | 133.5252 | | 232.3339 | 175.72 | | 0 | | |

[symbol]

indicates data missing or illegible when filed

### Example 6. Inducement of Mesophase by Stretching and Crimping

### Materials and Methods

A solution containing 500 mg of polycaprolactone dissolved in 1 ml of dichloromethane was prepared. To achieve complete dissolution, a series of three sequential cycles involving ultrasonication for 5 minutes and vortexing for 1 minute were done. Subsequently, the solution was poured into a Petri dish to form a film, which was left to dry overnight. The resulting film was subjected to X-ray diffraction (XRD) analysis.

### Results

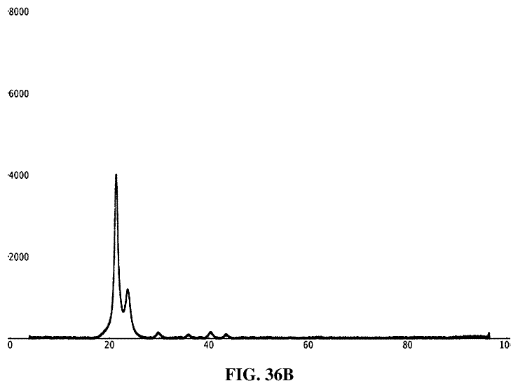

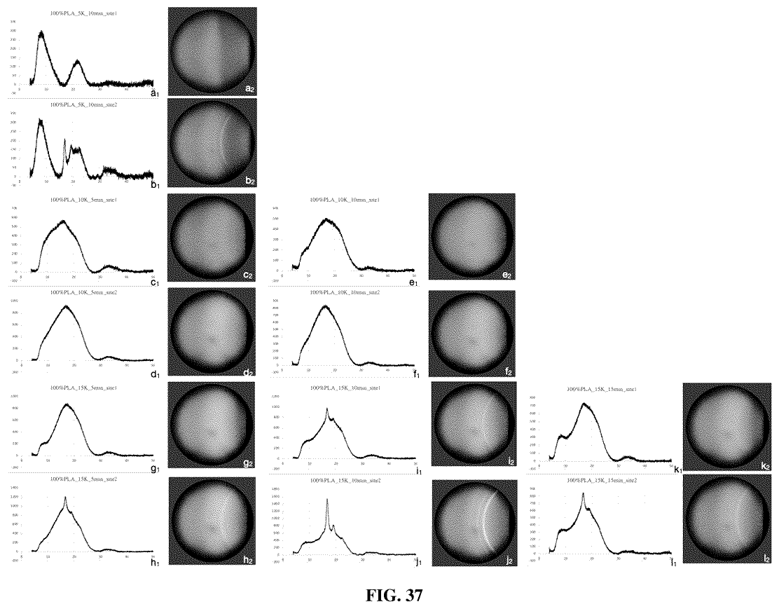

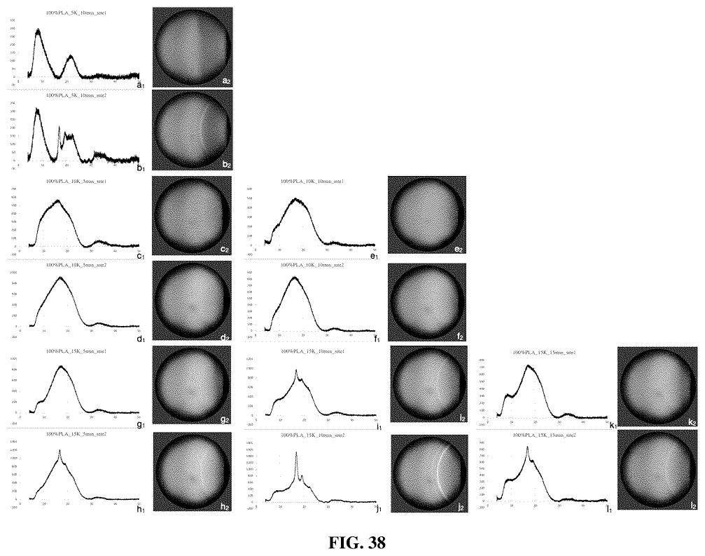

To investigate the effect of stretching the film, a segment measuring approximately 1 cm in width and 8 cm in length was carefully excised. This segment was then subjected to stretching until it reached its breaking point, utilizing an Instron tensile tester. Following the stretching, the segment was preliminarily examined to assess mesophase induction using polarized light microscopy. The images obtained from polarized light microscopy clearly exhibited birefringence at the location of fracture within the segment (data not shown). These observations were subsequently corroborated by conducting XRD analysis and comparing the obtained results with the XRD data obtained from the segment prior to its stretching using the Instron tensile tester. A broadening of the bands was observed in the stretched area which indicates the presence of a mesophase (FIGS. 36A and 36B).

To examine the impact of crimping on the induction of mesophase, a scientific study was conducted using polycaprolactone rods. Polycaprolactone was extruded from a 10 ml syringe into a tube at 65° Celsius to make a cylindrical rod. This rod was then crimped using a commercially available crimpling tool such that there were segments of crimped and uncrimped regions. Birefringence was notably observed using polarized light microscopy in the crimped regions (data not shown).

### Example 7: Preparation of Curcumin and PLA Loaded Films

### Methods

An ester-terminated Poly(L-lactide) (Durect Lactel; Part #B6002-2P, and Lot #A12-039a) was used. Its inherent viscosity range was 0.9-1.2 dL/g in ChCl₃. The solvent used was Dichloromethane (CH₂Cl₂), procured from Sigma-Aldrich, containing 40-150 ppm amylene as a stabilizer. It had a density of 1.325 g/mL at 25° C. and a molecular weight of 84.93 g/mol.

In the film casting process, 250 mg of PLA was precisely weighed and transferred into a 50 mL disposable glass vial. 2 mL of dichloromethane (DCM) was then added to the vial using a glass pipette. AAn Aquasonic Model 150T sonicator and Talboys™ vortex was used to ensure a homogeneous PLA mixture.

The mixing process for PLA and DCM was a two-step procedure, repeated twice. First, the tightly capped vials were placed onto the sonicator machine's rack. The water in the machine was filled just above the vial's liquid level. After 5 minutes of sonication, the vials were removed, the water from the glass surface was wiped (for better grip during vortexing), and the vials were vortexed at 5,000 rpm for another 5 minutes. This process was repeated once more.

Afterward, the mixture was slowly poured onto a KIMAX® COVER Kimble glass petri dish with a 6 cm diameter. It's worth noting that pouring the liquid gently in the center of the dish and carefully tilting it to distribute the liquid resulted in a more homogeneous film than randomly pouring. The film was allowed to dry for 24 hours before peeling it off the dish.

A Specac hand press machine was used for first pressing. During this process, the dried film was folded in half four times, then the smaller tipped end was inserted into the pressing die, following the user manual to apply the desired pressure.

### Results

FIG. 37 illustrates the XRD plots and 2D-XRD images of hand-pressed, film-casted samples made from 100% Poly(L-lactide) (PLA) at room temperature under varying pressures and durations: 5,000 lbs, for 10 minutes; 10,000 lbs, for 5 and 10 minutes; and 15,000 lbs, for 5, 10, and 15 minutes. “Site 1” and “Site 2” denote XRD files generated from scanning two arbitrary points on the hand-pressed tablets. FIG. 37 (see c₁, to g₂) demonstrates the greatest sign of induction of liquid crystals due to the saturated halo rings in the middle of the 2D-XRD captures, as well as the broad and medium-intensity peaks on the XRD files. FIG. 37 (see a₁ and a₂) indicate the amorphous state of the film, due to broad and low-intensity peaks on the XRD files, and the vastly empty 2D-XRD captures. FIG. 37 (see i₂-l₂) indicates the induction of not only liquid crystals but also condis crystals, due to the occurrence of sharper curves on the 2D-XRD captures, and the narrowing of peaks on the XRD files.

FIG. 38 display XRD plots and 2D-XRD images of hand-pressed, film-casted samples of 1% Curcumin and 99% PLA (1% Cur_99% PLA) at room temperature under various pressures and durations: 5,000 lbs, for 5 and 10 minutes; 10,000 lbs, for 5 and 10 minutes; and 15,000 lbs, for 5, 10, and 15 minutes. The 1% Cur_99% PLA hand-pressed tablets were produced from film-casted mixtures of 2.5 mg Curcumin (purchased from SIGMA-ALDRICH, Co., tag C1386-10G, Lot #SHBN9867, molecular weight 368.38 g/mol, melting point 175° C.), 247.5 mg PLA, and 2 mL of DCM. An induction of highly ordered state, which is likely to be the condis crystals (see a₁ and a₂ of FIG. 38). When duration of 5,000 lbs. of pressure extended to 10 minutes; b₁ and b₂ of FIG. 38 indicate the reduction of condis crystals, and the gain of liquid crystal form. FIG. 38 c₁, c₂, di, and d₂ of show good inductions of liquid crystals when pressing pressure increased from 5,000 lbs. to 10,000 lbs. From FIG. 38 g₁-l₂, an increase in the condis crystal state is represented by the emergence of sharper peaks on the XRD files, and the narrowing and elevating PLA peak heights. This example demonstrates that different types of mesophase can be induced with the same loading of drug while applying low pressure at room temperature.

### Example 8: Release of Dolutegravir (DTG) from Mesophase-Induced Polymer Tablets

### Methods

Dolutegravir (DTG) was the drug of interest, and it was incorporated into different mixtures at concentrations of 1%, 5%, 30%, and 50% relative to polycaprolactone with the total weight of mixture being 250 mg. Briefly, 2.5 mg, 12.5 mg, 75 mg and 125 mg of dolutegravir were weighed and the weight was adjusted to a total of 250 mg using polycaprolactone (PCL). To ensure solubility of the DTG-PCL mixtures in dichloromethane (DCM), the DTG-PCL mixtures were subjected to three cycles of vortexing (2 minutes each) and sonication (5 minutes each). Visual inspection of the solution confirmed that there were no solids remaining and that all the solids had been solubilized in DCM.

Subsequently, the DTG-PCL mixtures were cast onto plates and allowed to air dry overnight, resulting in the formation of thin films, referred to as Film Casts. These films were then subjected to compression to form tablets. In the first press, the samples were pressed at 10,000 lbs for 5 minutes at 25° C. to compact the materials. The second press was performed using a Carver press at room temperature, applying 10,000 lbs of pressure for another 5 minutes. The final dimensions of the second press tablets are described in Table 4.

TABLE 4
Dimensions of 1%, 5%, 30% and 50% loaded drug tablets

| Tablet | Diameter 1 (mm) | Diameter 2 (mm) | Thickness (mm) |
|---|---|---|---|
| 1% | 21.25 | 21.23 | 0.61 |
| 5% | 20.37 | 20.51 | 0.84 |
| 30% | 20.3 | 20.32 | 0.79 |
| 50% | 18.5 | 19.12 | 0.76 |

To investigate drug release, the tablets obtained from the second press were placed in 200 mL of phosphate-buffered saline (PBS) with a pH of 7.4 and kept at 37° C. At predetermined time intervals, 2-mL aliquots were withdrawn from the release media for drug concentration analysis. To maintain sink conditions, the entire release media was replaced with fresh PBS after each 24-hour interval.

Quantitative analysis of DTG release was conducted using High-Performance Liquid Chromatography (HPLC). The HPLC method involved a runtime of 3 minutes and employed a Waters Xbridge BEH C18 2.5 μm 4.6×100 mm column at room temperature. A 10-μL volume of the sample was injected, and the mobile phase composed of a mixture of water and acetonitrile (20:80).

### Results

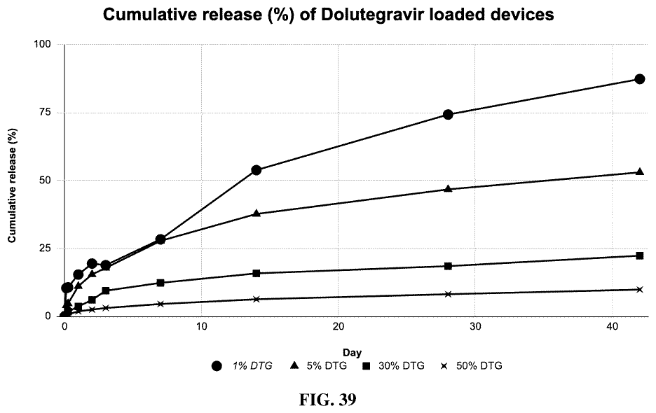

A linearity curve was first developed with an R² of 0.998 (linear graph not shown), as well as a chromatograph, both using HPLC, (chromatogram not shown). The retention time, area under curve, and peak height for DTG were 1.268 minutes, 939924 AU-minutes, and 122757 AU, respectively. An analysis of drug release samples was then performed (FIG. 39). At the end of 6 weeks, 87.27%, 52.95%, 22.30% and 9.83% of DTG were released from 1%, 5%, 30% and 50% loaded tablets respectively. Lower loading tablets release the hydrophobic drug more rapidly compared to higher loaded tablet. The 1% dolutegravir-loaded tablet released 87% of the drug within 6 weeks compared to 9.38% drug released over 6 weeks for the 50% loaded tablet.

Those skilled in the art will recognize, or be able to ascertain using no more than routine experimentation, many equivalents to the specific embodiments of the invention described herein. Such equivalents are intended to be encompassed by the ensuing claims.

We claim:

1. A composition comprising a polymer matrix comprising a (i) hydrophobic polymer and/or an amphiphilic polymer and (ii) a hydrophobic drug, wherein the polymer matrix has a mesophase content between about 5% and about 50% as measured using X-ray diffraction techniques at atmospheric pressure and room temperature or differential scanning calorimetry at a range of temperatures below the melting temperature of the hydrophobic polymer or amphiphilic polymer,
wherein the amphiphilic polymer comprises a hydrophobic polymer segment and a hydrophilic polymer segment.

2. The composition of claim 1, wherein the hydrophobic polymer and/or the hydrophobic polymer segment is a biodegradable polymer, optionally a biodegradable thermoplastic polymer.

3. The composition of claim 1, wherein the hydrophobic polymer and/or hydrophobic polymer segment are selected from the group consisting of a polyester, polyanhydride, poly(ortho) esters, poly(p-dioxanone), poly(polyurethane), polycarbonate, and a combination thereof.

4. The composition of claim 1, wherein the hydrophobic polymer and/or hydrophobic polymer segment is a polyester, optionally a linear aliphatic polyester.

5. The composition of claim 1, wherein:
(ai) the hydrophobic polymer and/or hydrophobic polymer segment is selected from the group consisting of a poly(lactic acid), poly(caprolactone), poly(lactic acid-co-glycolic acid), poly(pentadecalactone), poly(hydroxybutyrate-co-hydroxyvalerate), poly(hydroxybutyrate), polybutylene succinate, and a combination thereof; or
(aii) the hydrophilic polymer segment comprises poly(ethylene oxide), poly(ethylene glycol), poly(vinyl alcohol), poly(aminoethylmethacrylate), poly(hydroxy ethylmethacrylate), poly(acrylonitrile), or poly(amide).

6. The composition of claim 1, wherein:
(ai) the hydrophobic drug is homogeneously distributed within the polymer matrix; or
(aii) the hydrophobic drug is not covalently bound to the hydrophobic polymer.

7. The composition of claim 1, wherein the hydrophobic drug has a loading between about 1% w/w and about 50% w/w.

8. The composition of claim 1, wherein the hydrophobic drug is released from the composition at least one day.

9. The composition of claim 1, wherein the polymer matrix is formed by a method comprising:
(i) exposing a combination of the hydrophobic polymer and the hydrophobic drug to a first temperature and a pressure greater than atmospheric pressure.

10. The composition of claim 9, wherein:
(ai) the first temperature is a temperature less than the melting temperature of the polymer;
(bi) prior to step (i), the combination is heated to the first temperature; or
(ci) after step (i), the mixture is cooled while maintaining the pressure attained at step (i).

11. The composition of claim 1, wherein the composition is in the form of capsules, tablets, troches, or implants, optionally wherein the composition is in a solid form.

12. The composition of claim 1, further comprising an excipient dispersed within or associated with the polymer matrix, preferably wherein the excipient is a plasticizer, stabilizer, or drug-release altering agent.

13. A method of administering a hydrophobic drug to a subject in need thereof, the method comprising implanting or injecting into the subject the composition of claim 1.

14. A method of inducing a mesophase content in a polymeric matrix comprising a hydrophobic polymer and/or amphiphilic polymer, and a hydrophobic drug, the method comprising:
(A):
(i) heating a combination of the hydrophobic polymer and/or amphiphilic polymer, and the hydrophobic drug to a first temperature,
(ii) applying to the combination an elevated pressure greater than atmospheric pressure and at the first temperature, and
(iii) cooling the combination to a desired temperature, or
(B)
(bi) film casting a combination of the hydrophobic polymer and/or amphiphilic polymer, and the hydrophobic drug to form a film, and
(bii) stretching the film until it reaches its breaking point,
wherein the amphiphilic polymer comprises a hydrophobic polymer segment and a hydrophilic polymer segment.

15. The method of claim 14:
(ai) comprising applying to the combination a first pressure prior to step (i); or
(aii) further comprising step (iv) assessing the mesophase content at a second temperature below the first temperature, using X-ray diffraction techniques at atmospheric pressure.

16. The method of claim 15, wherein the first pressure is greater than about 13 MPa.

17. The method of claim 14, wherein the first temperature is a temperature below the melting temperature of the hydrophobic polymer and/or the amphiphilic polymer.

18. The method of claim 15, wherein the second temperature is room temperature.

19. The method of claim 14, wherein:
(ai) step (ii) is performed within a time between about 1 min and about 20 mins,
(aii) step (iii) is performed while maintaining the combination at the elevated pressure, or
(aiii) after step (iii), the polymer matrix has a mesophase content between about 5% and about 50% as measured using X-ray diffraction techniques at atmospheric pressure.

20. The method of claim 14, wherein the elevated pressure is greater than about 13 MPa.

21. The method of claim 14, wherein hydrophobic polymer and/or hydrophobic polymer segment comprises a biodegradable polymer, optionally a biodegradable thermoplastic polymer.

22. The method of claim 14, wherein the hydrophobic polymer and/or hydrophobic polymer segment is selected from the group consisting of a polyester, polyanhydride, poly(ortho) esters, poly(p-dioxanone), poly(polyurethane), polycarbonate, and a combination thereof.

23. The method of claim 14, wherein the hydrophobic polymer and/or hydrophobic polymer segment is a polyester, optionally a linear aliphatic polyester.

24. The method of claim 14, wherein:
(ai) the hydrophobic polymer and/or hydrophobic polymer segment is selected from the group consisting of a poly(lactic acid), poly(caprolactone), poly(lactic acid-co-glycolic acid), poly(pentadecalactone), poly(hydroxybutyrate-co-hydroxyvalerate), poly(hydroxybutyrate), polybutylene succinate, and a combination thereof; or
(aii) the hydrophilic polymer segment comprises poly(ethylene oxide), poly(ethylene glycol), poly(vinyl alcohol), poly(aminoethylmethacrylate), poly(hydroxy ethylmethacrylate), poly(acrylonitrile), or poly(amide).
